(19) United States
(12) Patent Application Publication
(10) Pub. No.: US 2020/0179488 A1
(43) Pub. Date: Jun. 11, 2020

(54) Secretin Receptor Agonists to Treat Diseases or Disorders of Energy Homeostasis

(71) Applicant: Technische Universität München, München (DE)

(72) Inventors: Martin KLINGENSPOR, Freising (DE); Sarah-Madeleine GABLER, München (DE); Yongguo LI, Freising (DE)

(21) Appl. No.: 16/303,775

(22) PCT Filed: May 23, 2017
(86) PCT No.: PCT/EP2017/062420

§ 371 Date: Nov. 21, 2018

(30) Foreign Application Priority Data
May 23, 2016 (EP) 16170863.1

(51) Int. Cl. A61K 38/22; A61K 8/64; A61Q 19/06; G01N 33/50

(52) U.S. Cl. CPC A61K 38/2242; A61K 8/64; A61K 45/06; G01N 33/5044; A61Q 19/06

(57) ABSTRACT

The present invention relates to a secretin receptor modulator for use in the prevention and/or treatment of a disease or disorder of energy homeostasis, wherein (a) said secretin receptor modulator is a secretin receptor agonist and said disease or disorder is obesity, dyslipidemia, diabetes, insulin resistance, hyperglycemia, high blood pressure or metabolic syndrome, whereby the secretin receptor agonist increases non-shivering thermogenesis in brown adipocytes and/or increases the expression of uncoupling protein 1 (UCP1) in brown adipocytes and/or decreases food intake in a UCP1-dependent manner resulting in the prevention and/or treatment of said disease or disorder; or (b) said secretin receptor modulator is a secretin receptor antagonist and said disease or disorder is cachexia. The invention further relates to a method of increasing non-shivering thermogenesis in brown adipocytes and/or increasing the expression of uncoupling protein 1 (UCP1) in brown adipocytes, to a method of decreasing non-shivering thermogenesis in brown adipocytes, to a method of identifying a secretin receptor agonist capable of increasing non-shivering thermogenesis in brown adipocytes and/or increasing the expression of uncoupling protein 1 (UCP1) in brown adipocytes, to a method of identifying a secretin receptor antagonist capable of decreasing non-shivering thermogenesis in brown adipocytes, to the use of the secretin receptor for screening (a) for secretin receptor agonists that increase non-shivering thermogenesis in brown adipocytes and/or increase the expression of uncoupling protein 1 (UCP1) in brown adipocytes and/or decrease food intake in a UCP1-dependent manner; and/or (b) for secretin receptor antagonists that decrease non-shivering thermogenesis in brown adipocytes, and to the use of a secretin receptor agonist to activate non-shivering thermogenesis in brown adipocytes and/or to increase the expression of uncoupling protein 1 (UCP1) in brown adipocytes and/or to decrease food intake in a UCP1-dependent manner for reducing body weight for cosmetic purposes as well as to the use of a secretin receptor antagonist to decrease thermogenesis in brown adipocytes for increasing body weight for cosmetic purposes.

The present invention relates to a secretin receptor modulator for use in the prevention and/or treatment of a disease or disorder of energy homeostasis, wherein (a) said secretin receptor modulator is a secretin receptor agonist and said disease or disorder is obesity, dyslipidemia, diabetes, insulin resistance, hyperglycemia, high blood pressure or metabolic syndrome, whereby the secretin receptor agonist increases non-shivering thermogenesis in brown adipocytes and/or increases the expression of uncoupling protein 1 (UCP1) in brown adipocytes and/or decreases food intake in a UCP1-dependent manner resulting in the prevention and/or treatment of said disease or disorder; or (b) said secretin receptor modulator is a secretin receptor antagonist and said disease or disorder is cachexia. The invention further relates to a method of increasing non-shivering thermogenesis in brown adipocytes and/or increasing the expression of uncoupling protein 1 (UCP1) in brown adipocytes, to a method of decreasing non-shivering thermogenesis in brown adipocytes, to a method of identifying a secretin receptor agonist capable of increasing non-shivering thermogenesis in brown adipocytes and/or increasing the expression of uncoupling protein 1 (UCP1) in brown adipocytes, to a method of identifying a secretin receptor antagonist capable of decreasing non-shivering thermogenesis in brown adipocytes, to the use of the secretin receptor for screening (a) for secretin receptor agonists that increase non-shivering thermogenesis in brown adipocytes and/or increase the expression of uncoupling protein 1 (UCP1) in brown adipocytes and/or decrease food intake in a UCP1-dependent manner; and/or (b) for secretin receptor antagonists that decrease non-shivering thermogenesis in brown adipocytes, and to the use of a secretin receptor agonist to activate non-shivering thermogenesis in brown adipocytes and/or to increase the expression of uncoupling protein 1 (UCP1) in brown adipocytes and/or to decrease food intake in a UCP1-dependent manner for reducing body weight for cosmetic purposes as well as to the use of a secretin receptor antagonist to decrease thermogenesis in brown adipocytes for increasing body weight for cosmetic purposes.

In this specification, a number of documents including patent applications and manufacturer's manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

Obesity is a medical condition in which excess body fat has accumulated to an extent that it has an adverse effect on health, leading to reduced life expectancy and/or increased health problems. For example, obesity is associated with various metabolic disturbances, such as type 2 diabetes, insulin resistance, hyperglycemia, dyslipidemia, high blood pressure and metabolic syndrome. Obesity is most commonly the consequence of a misbalanced energy homeostasis caused by a misbalance of food energy resorption and energy expenditure (e.g. basal metabolic rate, thermal effect of food, thermoregulatory heat production, physical activity and external work). However, the misbalanced energy homeostasis leading to obesity can also be the result of genetic susceptibility, endocrine disorders, neuroendocrine disorders, and/or medications.

Diseases of energy homeostasis such as obesity and secondary diseases of obesity (like dyslipidemia, type 2 diabetes, insulin resistance, hyperglycemia, high blood pressure and metabolic syndrome) are considered a major health issue. For example, obesity has emerged as a global health problem with more than 1.1 billion adults to be classified as overweight or obese (Oh, Curr Top Med Chem (2009), 9: 466-481). Nevertheless, treatment of diseases of energy homeostasis (such as obesity) is so far not satisfactory.

Mammals have two types of adipose tissue (i.e. fat), brown adipose tissue (BAT, also called brown fat, comprising brown adipocytes) and white adipose tissue (WAT, also called white fat, comprising white adipocytes and brite adipocytes). The primary function of brown adipose tissue is to produce body heat in mammals without the necessity to shiver. In brown adipose tissue, body heat is produced by signaling the mitochondria to allow protons to run back along the proton gradient without producing ATP (proton leak). This is realized by the uncoupling protein 1 (UCP1 or thermogenin) which allows re-entry of protons from the intermembrane space into the matrix, thereby bypassing ATP synthase, and thus, uncoupling oxygen consumption from ATP production. This alternative route for protons uncouples oxidative phosphorylation and releases energy as heat.

Brown adipose tissue is highly specialized for non-shivering thermogenesis. For example, as compared to white adipocytes, brown adipocytes have a higher number of mitochondria and these mitochondria have a high concentration of UCP1 in the inner membrane. The term “brown adipocytes” refers to all types of thermogenic, UCP1 expressing and/or multilocular cells. These are sometimes categorized into “classical brown” versus “beige” or “brite” and others.

Brown adipocytes are not restricted to uniform, classical BAT depots but are often found interspersed in white adipose tissue (WAT) depots. This second type of brown adipocyte has been termed beige or brite (brown in white) and seems to emerge from a different progenitor cell than classical brown fat cells (reviewed in (Pfeifer & Hoffmann, 2014)). To convert WAT into BAT by means of recruiting brite cells offers a possibility to massively increase the BAT amount accessible to therapeutic activation and at the same time decreases the amount of WAT, thereby replacing an energy-storing organ with an energy-dissipating one. This browning of white fat has been subject to intense research during the last years and several systemic interventions have been identified which at least to a certain degree increase the number of brite cells in mice. These systemic interactions include cold exposure and treatment with β-adrenergic agonists, cardiac natriuretic peptides or fibroblast growth factor 21 (FGF21) (Bordicchia et al., 2012; Fisher et al., 2012; Guerra, Koza, Yamashita, Walsh, & Kozak, 1998; Young, Arch, & Ashwell, 1984). However, the treatment with sympathomimetic drugs, e.g. beta3-adrenergic receptor agonists, is associated with detrimental cardiovascular side effects, e.g. elevated blood pressure and heart rate (Carey A L, et al. Diabetologia. 2013 January; 56(1):147-55. doi: 10.1007/s00125-012-2748-1).

Brown adipose tissue is especially abundant in newborns and in hibernating mammals. However, this tissue and its ability to combust nutrient energy into heat has recently gained increased attention by the scientific community after the repeated and convincing demonstration that adult healthy humans possess appreciable amounts of metabolically active BAT. It is accepted in the art that physiological or pharmacological activation of BAT thermogenesis is effective in treating some of the most widespread diseases of our time including obesity, dyslipidemia, diabetes, insulin resistance, hyperglycemia and metabolic syndrome.

The thermogenic function of brown adipose tissue (BAT) has been known for more than 50 years and has been subject to intense study. The specific properties of this tissue and its utility in the treatment of human metabolic disease are well accepted in the field and documented in numerous studies. A recent review stated that “from the literature, it is clear that expansion or increased activity of BAT in rodents is associated with a metabolically healthy phenotype.” (Lidell, (2014) J Int Med 276, 364-377). The presence of BAT is associated with low body mass index, low total adipose tissue content and a lower risk of type 2 diabetes mellitus (Lidell, loc. cit.). Thus, it is accepted in the art that expansion or increased activity of BAT is associated with a healthy phenotype in animals and adult humans. Vice versa, it is accepted that a defect in BAT in animals and adult humans is associated with or is a cause of metabolic disorders like diabetes and obesity (Himms-Hagen (1979) CMA Journal 121, 1361-1364; Rothwell (1979) Nature 281, 31-35). Indeed, recent studies documented the presence of substantial amounts of metabolically active brown adipose tissue in healthy adult humans, while BAT was significantly reduced in overweight or obese adult humans (Saito (2009) Diabetes; Lichtenbelt (2009) NEJM 360, 1500-1508, Virtanen (2009) NEJM 360, 1518-1525).

It is evident that the limiting factor for the therapy of metabolic diseases and disorders is the low amount of brown adipose tissue in adult, especially overweight, humans (Bartelt & Heeren, (2014) Nat Rev Endocrinol, 10(1), 24-36; Klingenspor, M., Fromme, T. (2012). Brown adipose tissue. In M. E. Symonds (Ed.), Adipose Tissue Biology (Vol. 414). Heidelberg: Springer; Saito et al., (2009) Diabetes, 58(7), 1526-1531; Virtanen et al., (2009) NEJM 360, 1518-1525). Animal experiments have indeed demonstrated that metabolic diseases and disorders like diabetes type I and II, obesity and hyperlipidemia can be treated by increasing the amount of active BAT (e.g. by converting white adipocytes into brown adipocytes); further it is accepted in the art that corresponding results can be obtained in therapy of humans suffering from metabolic diseases and disorders (Cinti (2006) Nutr Metabol Cardiovas Dis 16, 569-574; Bartelt (2011) Nature Medicine doi:10.1038/nm.2297); Gunawardana (2012) Diabetes 61, 674-682; Gunawardana (2014) World J Diabet 15, 420-430; Roman (2014) Translational Research, 1-15).

The properties of active brown adipose tissue that constitute its potential to counteract specific metabolic diseases are outlined in the following:

In relation to obesity and related disorders it is accepted that brown adipose tissue is characterized by its ability to release chemical energy as heat. Specific heat production is enormous and ranges from approx. 150 to 500 Watt/kilogram tissue in rodents. Increasing the mass of active brown adipose tissue necessarily leads to increased total energy expenditure and therefore to weight loss, assuming no compensation by increased food intake or decreases in other components of energy expenditure, such as metabolic rate in tissues other than brown fat or physical activity. The possibility to utilize active brown fat to counter obesity is generally accepted in the field and is a consequence of fundamental thermodynamics (Himms-Hagen, 1979; Klingenspor, 2012; Rothwell & Stock, 1979).

In relation to diabetes it is known that brown adipose tissue imports and combusts large amounts of glucose from the blood stream. In fact, the first unambiguous demonstration of active brown fat in humans relied on a high uptake of labelled glucose and is thus a certain property of human brown fat (van Marken Lichtenbelt et al., 2009; Virtanen et al., 2009). This property can be applied to counter excessive glucose levels that are the primary manifestation of diabetes. In a rodent model, a brown adipose tissue transplant into a white adipose tissue depot even effectively cures diabetes (Gunawardana & Piston, 2012).

The following non-limiting references support the utility of BAT in the therapy of metabolic diseases and disorders, like obesity and diabetes as well as related disorders, such as insulin-resistance, hyperglycemia and metabolic syndrome. For example, Lidell et al. (2014, loc. cit.) disclosed that “pharmacologic interventions that activate and expand BAT would provide a very attractive means for weight reduction, especially in individuals unable to exercise” and that “data from rodents provide robust indications for amelioration of insulin resistance upon physiological or pharmacological stimulation of BAT.” Gunawardana (2014, loc. cit) stated that “increasing the content of endogenous brown adipose tissue is known to combat obesity and type 2 diabetes in both humans and animal models.” Roman et al., (2014, loc. cit.) confirmed that “ultimately, the long-term effect of increasing BAT is expected to improve energy expenditure, leading to weight loss and increased insulin sensitivity” and “a BAT phenotype of the adipose organ in rodents is important for the prevention of obesity and diabetes. [ . . . ] in vivo and in vitro data suggest that the human adipose organ react similarly to the murine adipose organ.”

In relation to dyslipidemia, it is known in the art that the excessive metabolic rate of active brown adipose tissue is mainly fueled by lipids which are partially imported from the blood stream. In the active state, brown adipose tissue is considered the master regulator of triglyceride rich lipoprotein clearance and blood lipid abundance (Bartelt et al., 2011). From these established properties it is evident that increased mass and/or activity of active brown fat can be utilized to lower pathological levels of circulating lipoproteins and lipids. For example, Bartelt et al., (2011, loc. cit. disclose that “BAT activation is able to correct hyperlipidemia”. Bartelt & Heeren (2014, loc. cit.) described the following in relation to hyperlipidaemia:” The activation of brown adipose tissue (BAT), the primary organ for heat production, confers beneficial effects on adiposity, insulin resistance and hyperlipidaemia, at least in mice. As the amount of metabolically active BAT seems to be particularly low in patients with obesity or diabetes mellitus who require immediate therapy, new avenues are needed to increase the capacity for adaptive thermogenesis.” In view of the foregoing, it is evident that prevention and/or treatment of hypertrigliceridemia can be achieved by BAT activation.

Summarizing the above it is evident that an increase in active brown adipose tissue and/or an increase in its activity necessarily leads to an improvement in the primary manifestations of a disease or disorder of energy homeostasis, like obesity, diabetes, dyslipidemia, insulin resistance, hyperglycemia, high blood pressure and metabolic syndrome.

The amount of human BAT, however, is limited and estimated to account for approximately 0.05-0.1% of body mass as compared to a far more than 10-fold higher specific amount in mice. Thus, to therapeutically use the unique capabilities of BAT in humans, not only acute activators may be required, but also agents that recruit a greater number of brown adipocytes.

Clinical observations in cancer cachexia and experimental studies in animals support the view that hyperactive brown fat may contribute to wasting (see for example Petruzzelli et al, Cell Metab 2014; Tsoli et al, Semin Cell Dev Biol 2015). It has been hypothesized that tumour derived factors, such as PTHrP and IL-6, stimulate lipolysis in white and brown adipocytes as well as non-shivering thermogenesis in the latter. Antagonists, like beta adrenergic receptor antagonists, that inhibit PKA activation put a break on lipolytic activation (HSL phosphorylation) and thus inhibit substrate supply for mitochondrial fatty acid oxidation as the prime energy source for thermogenesis in brown adipocytes. This has been demonstrated to slow down the detrimental loss of adipose tissue mass in experimental mouse models for cachexia. Thus, it is evident that a decrease in the activity of brown adipose tissue necessarily leads to an improvement in the primary manifestations of cachexia.

The technical problem underlying the present invention was to identify means and methods for use in the prevention and/or treatment of a disease or disorder of energy homeostasis.

The solution to this technical problem is achieved by providing the embodiments characterized in the claims.

Accordingly, the present invention relates in a first embodiment to a secretin receptor modulator for use in the prevention and/or treatment of a disease or disorder of energy homeostasis, wherein (a) said secretin receptor modulator is a secretin receptor agonist and said disease or disorder is obesity, dyslipidemia, diabetes, insulin resistance, hyperglycemia, high blood pressure or metabolic syndrome, whereby the secretin receptor agonist increases non-shivering thermogenesis in brown adipocytes and/or increases the expression of uncoupling protein 1 (UCP1) in brown adipocytes and/or decreases food intake in a UCP1-dependent manner resulting in the prevention and/or treatment of said disease or disorder; or (b) said secretin receptor modulator is a secretin receptor antagonist and said disease or disorder is cachexia.

As such, described is also a method for the prevention and/or treatment of a disease or disorder of energy homeostasis, the method comprising administering an effective amount of a modulator of the secretin receptor to a subject in need thereof, wherein (a) said secretin receptor modulator is a secretin receptor agonist and said disease or disorder is obesity, dyslipidemia, diabetes, insulin resistance, hyperglycemia, high blood pressure or metabolic syndrome, whereby the secretin receptor agonist increases non-shivering thermogenesis in brown adipocytes and/or increases the expression of uncoupling protein 1 (UCP1) in brown adipocytes and/or decreases food intake in a UCP1-dependent manner resulting in the prevention and/or treatment of said disease or disorder; or (b) said secretin receptor modulator is a secretin receptor antagonist and said disease or disorder is cachexia.

The term “modulator of the secretin receptor” as used in accordance with the method of the invention relates to a given compound with the ability to modulate either directly or indirectly the secretin receptor so as to alter, i.e. increase or decrease, the biological function or activity of the secretin receptor. In other words, a compound “modulating” the secretin receptor alters the biological function or activity of the secretin receptor in that it increases or decreases said biological function or activity. For example, direct modulation may be achievable by binding to the secretin receptor, whereas indirect modulation may be achievable by interacting with molecules that are involved in the regulation of the secretin receptor, such as regulatory molecules upstream of the secretin receptor having an effect on the level of the secretin receptor ligand. Consequently, the modulator of the secretin receptor encompasses agonists of the secretin receptor (referred to herein as “secretin receptor agonists”) and antagonists of the secretin receptor (referred to herein as “secretin receptor antagonists”) which have a direct or indirect agonistic or antagonistic effect on the biological function or activity of the secretin receptor.

The term “secretin receptor” is well known in the art and refers to a G protein-coupled receptor belonging to the class B GPCR subfamily that binds to and is activated by the peptide hormone secretin. The human secretin receptor contains 440 amino acids and possesses seven transmembrane domains as it is classically known for G protein-coupled receptors. The mRNA is encoded in 13 exons and the transcript has a length of 1865 bps. The complete human protein and mRNA sequence of the secretin receptor are available in the databases maintained by the National Center for Biotechnology Information (NCBI), 8600 Rockville Pike, Bethesda Md., 20894 USA, under the accession numbers NP_002971.2 (GI: 114205383) for the protein and NM_002980.2 (GI: 114205382) for the mRNA, respectively, at the world wide web address: http://www.ncbi.nlm.nih.gov/.

Secretin is a classical gut hormone consisting of 27 amino acids and is predominantly found in the upper parts of the small intestine. The mRNA of secretin is encoded in 4 exons and the transcript has a length of 578 bps. The complete human protein and mRNA sequence of secretin are available in the databases maintained by the National Center for Biotechnology Information (NCBI), 8600 Rockville Pike, Bethesda Md., 20894 USA, under the accession numbers NP_068739.1 (GI: 11345450) for the protein and NM_021920.3 (GI: 992319647), respectively, at the world wide web address: http://www.ncbi.nlm.nih.gov/.

The binding of secretin to its receptor leads to the activation of an intracellular secondary messenger system, which triggers cellular processes. Like all members of the B family of GPCRs the secretin receptor activates the adenylate cyclase via the Gs protein. This leads to an accumulation of cAMP (Gether (2000), Endocrine reviews, 21: 90-113; Chow (1995), Biochemical and biophysical research communications, 212: 204-211) and the activation of protein kinase A (PKA). Moreover, the secretin receptor is also coupled to a Gq unit. This part of the pathway is activated when there are high concentrations of secretin and stimulates inositol triphosphate (IP 3), intracellular calcium and diacylglycerol (DAG) (Trimble et al. (1986), The Biochemical Journal, 239: 257-261; Trimble et al. (1987), Proceedings of the National Academy of Sciences of the United States of America, 84: 3146-3150; Neves et al. (2002), Science, 296: 1636-1639).

The known biological functions of secretin and its receptor are vast and constantly being expanded. They include numerous processes such as the stimulation of bile and bicarbonate secretion in the duodenum and in pancreatic and biliary ducts or the secretion of pepsin in the stomach to facilitate digestion (Otsuki et al. (1981), Digestive diseases and sciences, 26: 538-544; Friedman and Snape (1947), Fed Proc 6: 107; Christodoulopoulos et al. (1961), The American journal of physiology, 201: 1020-1024; Isenberg et al. (1984), Regulatory peptides, 8: 315-320; Jones et al. (1971), Gastroenterology, 60: 64-68; Sanders et al. (1983), The American journal of physiology, 245: G641-646). In addition, it is a matter of controversial discussion, if secretin is able to stimulate the secretion of insulin. An incretin effect seems more likely in mice and rats than in humans suggested by published results (Shima et al. (1978), Endocrinologia japonica, 25: 461-465; Ahren and Lundquist (1981), Diabetologia, 20: 54-59; Schaffalitzky de Muckadell (2001), Scandinavian journal of clinical and laboratory investigation Supplementum, 234: 105-108; Kraegen et al. (1970), The Journal of clinical investigation, 49: 524-529; Kofod (1986), Regulatory peptides, 15: 229-237). Secretin and also the secretin receptor have been detected in wide parts of the brain (O′Donohue et al. (1981), Proceedings of the National Academy of Sciences of the United States of America, 78: 5221-5224; Charlton et al. (1981), Peptides, 2 Suppl 1: 45-49; Mutt et al. (1979), Life sciences, 25: 1703-1707). Central actions of secretin are possibly related to fluid homeostasis (Chu et al. (2009), Proceedings of the National Academy of Sciences of the United States of America, 106: 15961-15966), control of social behavior and autistic disorders (Horvath et al. (1998), Journal of the Association for Academic Minority Physicians: the official publication of the Association for Academic Minority Physicians, 9: 9-15; Sandler et al. (1999), The New England journal of medicine, 341: 1801-1806), and the control of food intake in mice (Cheng et al. (2011), Neuropsychopharmacology, 36: 459-471). The secretin receptor is also expressed in white adipose tissue and has been shown to exhibit lipolytic action (Butcher and Carlson (1970), Acta physiologica Scandinavica, 79: 559-563). Especially in states of starvation elevated secretin and also glucagon levels increase the availability of free fatty acids to provide fuel for the metabolism in mice (Sekar and Chow (2014), Journal of lipid research, 55: 190-200).

Preferably, the secretin receptor is the human secretin receptor. The modulator of the human secretin receptor is, preferably, to be administered to a human subject in need of prevention and/or treatment of a disease or disorder of energy homeostasis. It is of note that the invention also explicitly encompasses any revised or corrected sequences of the secretin receptor or secretin which may be part of potential future updates in the database. Thus, the specification also accounts for future corrections and modifications in the entries of databases maintained, e.g. by the National Center for Biotechnology Information, which might occur due to the continuing progress of science.

A “patient” or “subject” for the purposes of the present invention includes both humans and other animals, particularly mammals. Thus, the methods are applicable to both human therapy and veterinary applications. In the preferred embodiment the patient or subject to be treated is a mammal (like pets, such as cats or dogs), and in the most preferred embodiment the patient is a human patient. The present invention also encompasses the medical intervention of diseases or disorders of energy homeostasis like obesity, dyslipidemia, diabetes, insulin resistance, hyperglycemia, high blood pressure or metabolic syndrome by use of the compounds, means and methods as described herein in a veterinary setting.

As known in the art, the term “energy homeostasis” relates to any process involved in the balance between energy intake (food consumption) and energy expenditure. In particular, energy homeostasis is the relation between intake of food and output of energy that is positive when the body stores extra food as fats and negative when the body draws on stored fat to provide energy for work. A significant contributor to energy expenditure of mammals is the generation of heat for keeping the body temperature constant as well as muscular and metabolic activities. In accordance with the invention, a disease or disorder of energy homeostasis is characterized by a positive energy homeostasis (wherein the body is storing energy as fat), namely obesity, dyslipidemia, diabetes, insulin resistance, hyperglycemia, high blood pressure and metabolic syndrome, or in the case of cachexia by a negative energy homeostasis. The terms “obesity”, “dyslipidemia”, “diabetes”, “insulin resistance”, “hyperglycemia”, “high blood pressure” and “metabolic syndrome” as used herein refer only to those forms of the respective disease or disorder that are caused or aggravated by a disturbed energy homeostasis. In view of the means and methods available for the diagnosis of the different diseases and disorders it is known in the art how to distinguish between forms caused and/or aggravated by misbalance in energy homeostasis and those independent thereof. For example, the term “high blood pressure” (also “hypertension”) as used herein refers only to forms that are caused or aggravated by a disturbed energy homeostasis. By contrast, treatment of other forms of high blood pressure such as those arising, for example, due to kidney or thyroid problems or congenital defects in blood vessels, is not envisaged in accordance with the present invention.

As used herein, the terms “treatment”, “treating”, “preventing” and “ameliorating” and the like generally mean obtaining a desired pharmacological and/or physiological effect. In the case of prevention, the effect may be prophylactic in terms of completely or partially preventing a disease or symptom thereof. In the case of treatment, the effect may be therapeutic in terms of partially or completely curing a disease and/or adverse effect attributed to the disease. Evidently, there may be cases where the treatment of one disease according to the invention will lead to the prevention of another disease that is associated with the treated disease. For example, the treatment of obesity can result in the prevention of diabetes. The term “treatment/treating” as used herein covers any treatment of a disease in a subject and includes: (a) inhibiting a disease or disorder of energy homeostasis, e.g. arresting its development; or (b) relieving a disease or disorder of energy homeostasis, e.g. causing regression of a disease or disorder of energy homeostasis. In accordance with the present invention, the term “prevention” or “preventing” of an disease means the disease per se can be hindered of developing or to develop into an even worse situation. Accordingly, it is one aspect of the present invention that the herein described modulators of the secretin receptor, preferably the secretin receptor agonists, can be employed in avoidance of a disease or disorder of energy homeostasis. It is understood that the modulation of the secretin receptor for the purpose of preventing a disease or disorder of energy homeostasis in accordance with the invention will be employed in subjects that are known to have predisposition to develop a disease or disorder of energy homeostasis described herein. For the diseases or disorders of energy homeostasis specifically identified herein, criteria exist for determining whether a subject has a predisposition to develop one or more of said disorders or diseases. For example, subjects that are predispositioned to develop type 2 diabetes are identified as prediabetic subjects by assessing, e.g., their fasting glucose levels. It is further understood that a preventive administration of a modulator of the secretin receptor in accordance with the invention is monitored to avoid occurrence of side effects and/or development of another disease or disorder of energy homeostasis. For example, the preventive administration of a secretin receptor agonist to prevent the development of obesity in a subject known to have a predisposition to develop obesity will not result in the development of cachexia in said subject. Establishing the right dosage and administration regimen for a corresponding preventive treatment can be done without further ado following routine methods known in the art.

Certain biological functions or activities of the secretin receptor known in the art have been described herein above in some detail. The present invention is, however, essentially based on the finding that the secretin receptor is specifically involved in a pathway that regulates energy metabolism. In accordance with the invention the biological function and activity of the secretin receptor that is relevant for the invention and to which is referred to herein, is the capability of the secretin receptor to regulate non-shivering thermogenesis, namely i) upon activation of the secretin receptor by an agonist to increase (the rate of) non-shivering thermogenesis in brown adipocytes and ii) upon inhibition of the secretin receptor by an antagonist to decrease (the rate of) non-shivering thermogenesis. A further biological function and activity of the secretin receptor that is relevant for the invention and to which is referred to herein is the capability to increase the expression of UCP1 in brown adipocytes upon activation by an agonist. Without being bound by a scientific theory, both biological functions and activities are considered to occur together upon activation of the secretin receptor by an agonist of the secretin receptor.

The term “brown adipocytes” as used herein refers to all types of thermogenic, UCP1 expressing cells. “Brown adipocytes” are in particular defined by expression of uncoupling protein 1 (herein also “UCP1”, “UCP-1”, “ucp-1”, “Ucp1” or “Ucp-1”, or thermogenin, i.e. abbreviations or synonyms as used in the art). The sequence and function of uncoupling protein 1 (UCP1) is well documented in the art and disclosed, inter alia, in Aquila et al. (1985) EMBO J 4(9):2369-2376; Bouillaud et al. (1986). J Biol Chem 261(4):1487-1490; Cassard et al., J Cell Biochem. 1990 July; 43(3):255-64; Nedergaard et al. Biochim Biophys Acta. (2001) March 1; 1504(1):82-106. Further, a second type of brown adipocyte exists that has been termed “beige” or “brite” (brown in white) as mentioned herein above. The developmental origins of such brite adipocytes are less clear. It has been shown that brite adipocytes stem from different progenitor cells than classical brown adipocytes (Seale P et al., Nature 2008; 454, 961-967). It was further shown that white adipocytes can convert into brite adipocytes that express UCP1 (Rosenwald et al., Nat Cell Biol. 2013 June; 15(6):659-67). Accordingly, brown adipocytes can be categorized into “classical brown” adipocytes versus “beige” or “brite” adipocytes. The term brown adipocytes as used herein is intended to encompass all of these adipocytes, namely “brown adipocytes in white adipose tissue” being synonym to both “beige” and “brite” and classical brown adipocytes. As such, one can also refer to “brown and brite” adipocytes. Moreover, brown adipocytes also encompass UCP1-expressing adipocytes found in skeletal muscle, or any other tissue harboring a subpopulation of UCP1-expressing adipocytes. To convert WAT into BAT by means of recruiting brite cells offers a possibility to massively increase the BAT amount.

Non-shivering thermogenesis is a means to produce heat in brown adipocytes as has been described herein above. More specifically, non-shivering thermogenesis is driven by lipolysis, a tightly regulated process involving enzymatic hydrolysis of stored triacylglycerol in adipocytes in response to physiological demands for maintaining body energy homeostatis. Lipolysis provides the needed free fatty acids (FFAs) as fuel for ATP production. Fatty acids that are released from lipolysis are also involved in heat production through β-oxidation and mitochondrial uncoupling leading to non-shivering thermogenesis in brown adipocytes. When a decreased body temperature triggered by cold exposure is sensed by the hypothalamus, catecholamines are released from the sympathetic nervous system, which activates adrenergic receptors of brown adipocytes which in turn stimulate the cAMP-dependent protein kinase PKA, leading to phosphorylation of hormone sensitive lipase (HSL) and thereby increased lipolysis. In this scenario, lipolysis can also be triggered by pharmacological adrenergic receptors agonist.

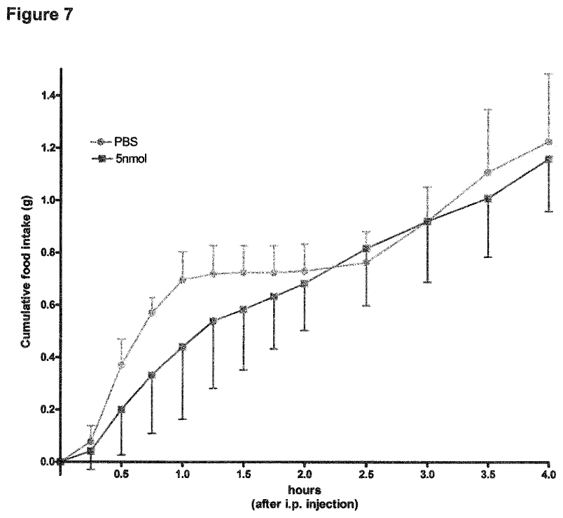

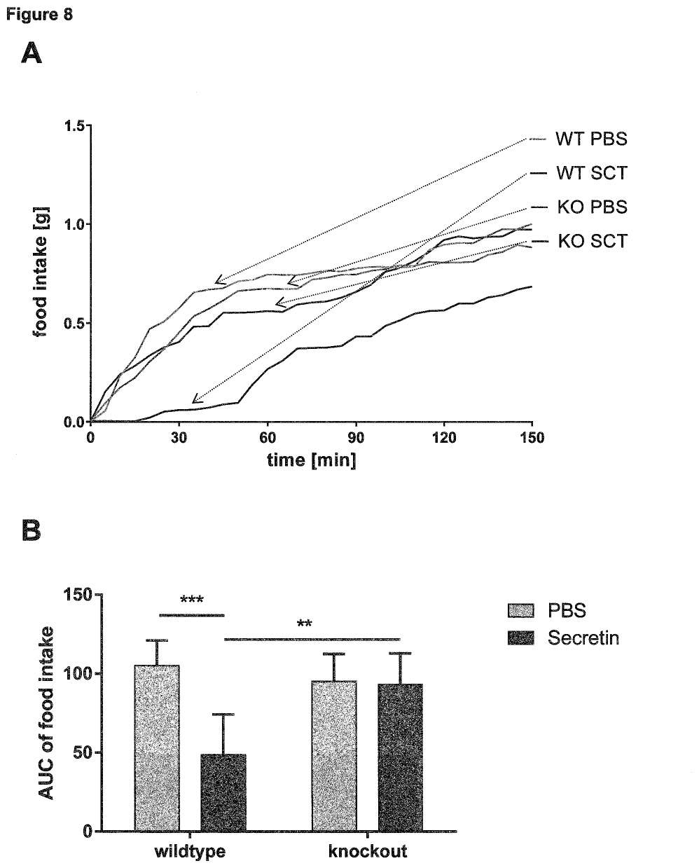

In accordance with one aspect of the invention, the biological function or activity of the secretin receptor is increased, namely non-shivering thermogenesis in brown adipocytes is increased and/or the expression of UCP1 in brown adipocytes is increased, by compounds designated “agonists” of the secretin receptor. As such, an agonist can also be referred to as “activator” of the secretin receptor. Additionally or alternatively, such agonists decrease food intake in a UCP1-dependent manner. Therefore, another biological function or activity of the secretin receptor is identified herein to be control of food intake. The term “UCP1-dependent manner” means that if UCP1 expression is increased via the action of the agonist on the secretin receptor, food intake is decreased in comparison to food intake without a secretin receptor agonist. As shown in the example section, the secretin receptor controls UCP1-expression. Example 8 (see also FIGS. 7 and 8) shows the decrease in food intake upon administration of secretin. Preferably, the decrease of food intake occurs during the initial phase, such as preferably less than 2 hours, more preferred less than 1.5 hours after fasting or, in other words, after starting a meal. The decrease in food intake after fasting is dependent on the bioavailability of the secretin receptor agonist. It is routine to assess the bioavailability of a given compound and adapt the administration regimen to achieve a consistent pharmaceutical effect. Thus, it will be possible to consistently decrease food intake using a secretin receptor agonist in accordance with invention. Increased caloric intake is one major contributor to the establishment of positive energy balance which results in increased fat storage and promotes the development of overweight and obesity causing metabolic dysregulation if maintained for longer time periods. Therapeutical interventions successfully reducing caloric intake are known to improve metabolic health in obese subjects. An agonist may perform any one or more of the following effects in order to increase the biological function or activity of the protein to be activated, i.e. the secretin receptor: (i) the transcription of the gene encoding the protein to be activated is increased, i.e. the level of mRNA is increased, (ii) the translation of the mRNA encoding the protein to be activated is increased, (iii) the protein performs its biochemical function with increased efficiency in the presence of the activator, and (iv) the protein performs its cellular function with increased efficiency in the presence of the activator.

Compounds falling in class (i) include compounds acting on the transcriptional machinery and/or its interaction with the promoter of said gene and/or with expression control elements remote from the promoter such as enhancers, so that the transcription of the secretin receptor is increased. Compounds of class (ii) comprise compounds that reduce the degradation rate of the protein to be activated, increase mRNA concentration (which may be identical with compounds of class (i)), and/or increase ribosome density. Compounds of class (iii) act on the molecular function of the protein to be activated, such as receptor signaling activity and activation of downstream target molecules, so that the secretin receptor protein performs its biochemical function with increased efficiency. Accordingly, active site binding compounds are envisaged, i.e. compounds that bind to the secretin receptor. Class (iv) includes compounds which do not necessarily bind directly to the target, but still influence its function or activity, for example by binding to and/or activating the function or increasing the expression of members of a pathway which comprises the target, i.e. the secretin receptor, so that the secretin receptor performs its cellular function with increased efficiency. These members may be upstream of the target within said pathway. For example, such compounds may alter, such as increase, the affinity or rate of binding of a known ligand to the receptor. Preferably, agonists belong to compounds falling under class (iii) and/or (iv), more preferred under class (iii). An example, of a secretin receptor agonist falling within class (iv) is an inhibitor of the angiotensin type I receptor (Agtr1). This receptor forms heterodimers with the secretin receptor and thereby inhibits the induction of cAMP responses by secretin stimulation of the secretin receptor (Lee et al., FASEB J. 2014 June; 28(6):2632-44. doi: 10.1096/fj.13-246868). Inhibiting said activity of Agtr1 is expected to lead to an increased biological function or activity of the secretin receptor, such as enhancing the increase UCP1-expression in response to secretin. Inhibition said activity of Agtr1 can, for example, be achieved by knockdown (using, e.g., RNAi based methods).

In accordance with the invention, the biological function or activity of the secretin receptor is decreased, namely non-shivering thermogenesis in brown adipocytes is decreased, by compounds designated “antagonists” of the secretin receptor. As such, an antagonist can also be referred to as an “inhibitor” of secretin receptor. A decrease can refer to a reduction or also a complete abolishment of said biological function or activity of the secretin receptor. An antagonist may perform any one or more of the following effects in order to reduce or abolish the biological function or activity of the protein to be inhibited, i.e. the secretin receptor: (i) the transcription of the gene encoding the protein to be inhibited is lowered, i.e. the level of mRNA is lowered, (ii) the translation of the mRNA encoding the protein to be inhibited is lowered, (iii) the protein performs its biochemical function with lowered efficiency in the presence of the antagonist, and (iv) the protein performs its cellular function with lowered efficiency in the presence of the antagonist.

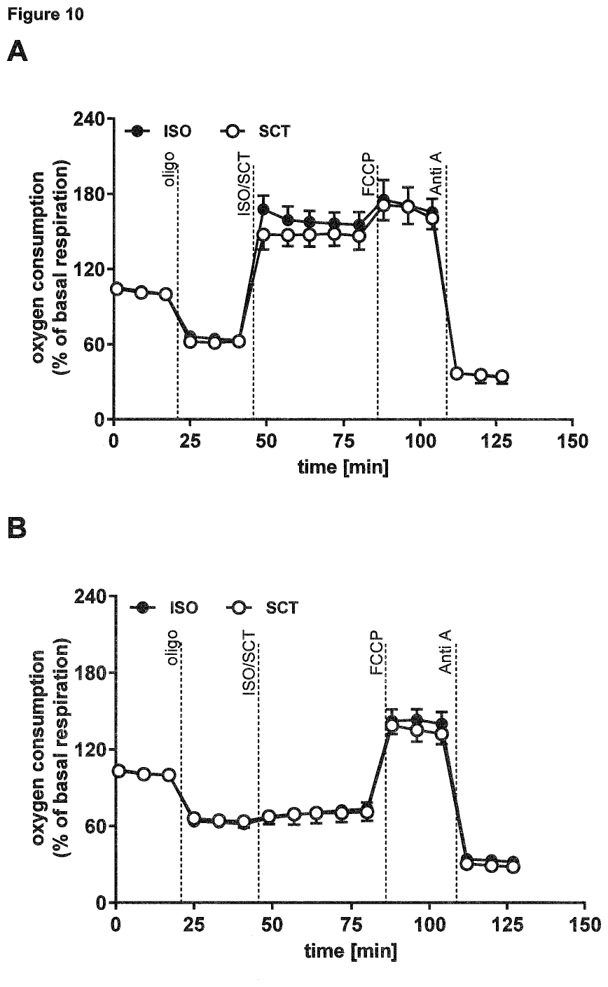

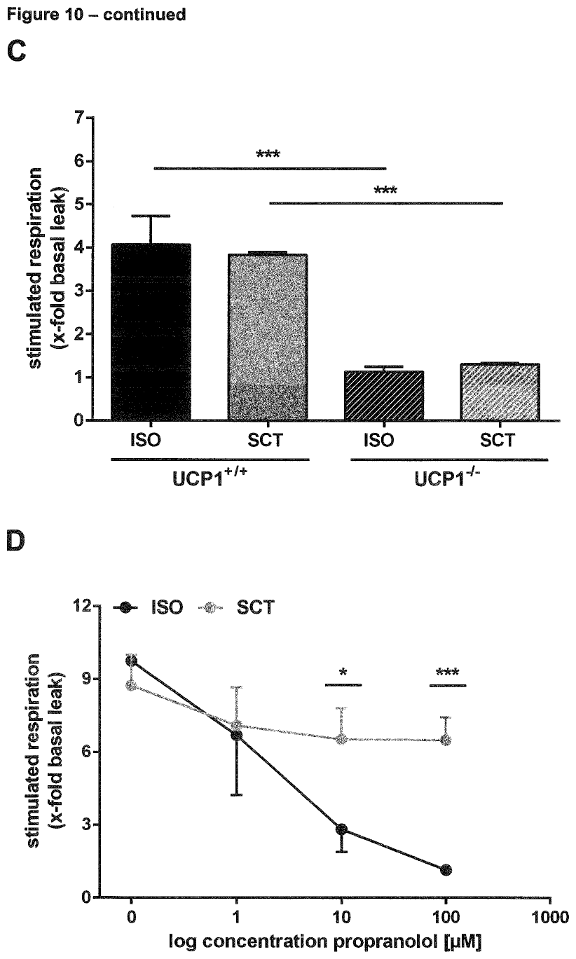

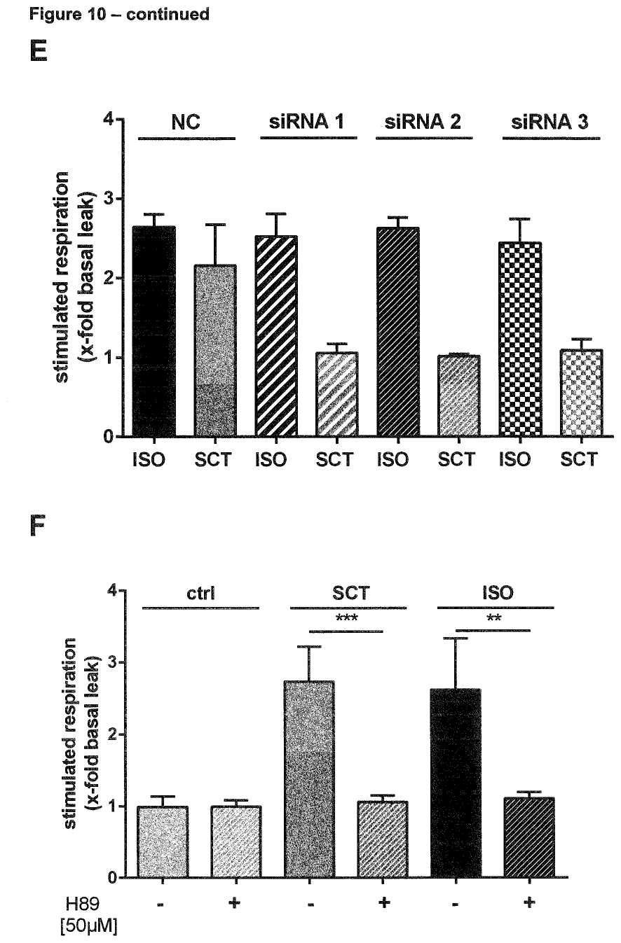

Compounds falling in class (i) include compounds interfering with the transcriptional machinery and/or its interaction with the promoter of said gene and/or with expression control elements remote from the promoter such as enhancers, so that the transcription of the secretin receptor is lowered. Compounds of class (ii) comprise antisense constructs and constructs for performing RNA interference (e.g. siRNA) well known in the art (see, e.g. Zamore (2001) Nat Struct Biol. 8(9), 746; Tuschl (2001) Chembiochem. 2(4), 239). The exemplary siRNAs against the secretin receptor of SEQ ID NOs 8, 9 and 10 have been shown to be effective, i.e. act as an antagonist/inhibitor (see FIG. 10E and example 10). Compounds of class (iii) interfere with the molecular function of the protein to be inhibited, such as receptor signalling activity and activation of downstream target molecules, so that the secretin receptor protein performs its biochemical function with lowered efficiency. Accordingly, active site binding compounds are envisaged which, e.g., competitively bind to the active site (such as e.g., an inactive ligand can be a secretin receptor antagonist). Class (iv) includes compounds which do not necessarily bind directly to the target, but still interfere with its function or activity, for example by binding to and/or inhibiting the function or inhibiting expression of members of a pathway which comprises the target, so that the secretin receptor performs its cellular function with lowered efficiency. These members may be upstream of the target within said pathway. For example, such compounds may alter, such as decrease, the affinity or rate of binding of a known ligand to the receptor or compete with a ligand for binding to the receptor or displace a ligand bound to the receptor. Preferably, antagonists belong to compounds falling under class (ii), (iii) and/or (iv), more preferred under class (iii). An example, of an antagonist falling within class (iii) is the angiotensin type I receptor (Agtr1). Increasing the activity of Agtr1 (e.g. by increasing its expression) is expected to lead to a decreased biological function or activity of the secretin receptor. An example of an antagonist falling within class (iv) is a protein kinase A inhibitor, such as, e.g., H89 (IUPAC name: N-[2-[[3-(4-Bromophenyl)-2-propenyl]amino]ethyl]-5-isoquinolinesulfonamide; Chemical formula: C₂₀H₂₀BrN₃O₂S; CAS number: 127243-85-0).

The agonist or antagonist, in accordance with the present invention, may in certain embodiments be provided as a proteinaceous compound or as a nucleic acid molecule encoding said agonist or antagonist. For example, the nucleic acid molecule encoding the agonist or antagonist may be incorporated into an expression vector comprising regulatory elements, such as for example specific promoters, and thus can be delivered into a cell. Methods for targeted transfection of cells and suitable vectors are known in the art, see for example Sambrook and Russel (“Molecular Cloning, A Laboratory Manual”, Cold Spring Harbor Laboratory, N.Y. (2001)). Incorporation of the nucleic acid molecule encoding the agonist or antagonist into an expression vector allows to either selectively or permanently elevate the level of the encoded agonist or antagonist in any cell or a subset of selected cells of the recipient. Thus, a tissue- and/or time-dependent expression of the agonist or antagonist can be achieved, for example, restricted to adipocytes. Suitable methods are known in the art and can, e.g., involve the use of the ap2 promoter, adiponectin promoter, UCP1 promoter, the adipocyte homing peptide (Won et al., Nature Materials 13, 1157-1164 (2014)) or the peptide motif CKGGRAKDC (SEQ ID NO: 1) that homes to white fat vasculature (Kolonin et al., Nature Medicine 10, 625-632 (2004)).

In a preferred embodiment, the antagonist decreases the biological function or activity, namely non-shivering thermogenesis in brown adipocytes, of the secretin receptor by at least 10%, such as at least 20%, at least 30%, at least 40% or at least 50%, preferably by at least 75%, more preferred by at least 90% and even more preferred by at least 95% such as at least 98% or even by 100% in comparison to said biological function or activity in the absence of said antagonist. The term reduction by at least, for example 75%, refers to a decreased biological function or activity such that the secretin receptor loses 75% of said biological function or activity and, consequently, has only 25% of said function or activity remaining as compared to the secretin receptor that is not inhibited. In the case of an agonist it is preferred that it increases the biological function or activity, namely non-shivering thermogenesis in brown adipocytes and/or the expression of UCP1 in brown adipocytes, of the secretin receptor by at least 10%, preferably at least 20% such as at least 30%, at least 40%, at least 50%, at least 60%, more preferred by at least 70%, such as at least 80%, at least 90%, at least 100%, and even more preferred by at least 150%, at least 200%, at least 500% or even 1000%. In the case of the biological function or activity of the secretin receptor to control food intake, it is preferred that “decrease” in food intake means a that the secretin receptor agonist decreases food intake by no more than 10%, such as no more than 20%, more preferred no more than 30%, such as no more than 40%, and most preferred no more than 50%.

The function of any of the agonists or antagonists referred to in the present invention may be identified and/or verified by using high throughput screening assays (HIS), where possible. High-throughput assays, independently of being biochemical, cellular or other assays, generally may be performed in wells of microtiter plates, wherein each plate may contain, for example 96, 384 or 1536 wells. Handling of the plates, including incubation at temperatures other than ambient temperature, and bringing into contact of test compounds with the assay mixture is preferably effected by one or more computer-controlled robotic systems including pipetting devices. In case large libraries of test compounds are to be screened and/or screening is to be effected within short time, mixtures of, for example 10, 20, 30, 40, 50 or 100 test compounds may be added to each well. In case a well exhibits biological activity, said mixture of test compounds may be de-convoluted to identify the one or more test compounds in said mixture giving rise to the observed biological activity.

The determination of binding of potential agonists or antagonists, e.g., to the secretin receptor, can be effected in, for example, any binding assay, preferably biophysical binding assay, which may be used to identify binding of test molecules prior to performing the functional/activity assay with the agonist or the antagonist. Suitable biophysical binding assays are known in the art and comprise fluorescence polarization (FP) assay, fluorescence resonance energy transfer (FRET) assay and surface plasmon resonance (SPR) assay.

In cases where the antagonist acts by decreasing the expression level or the agonist acts by increasing the expression level of the target protein, e.g., the secretin receptor or an activator thereof, the determination of the expression level of the protein can, for example, be carried out on the nucleic acid level or on the amino acid level.

Methods for determining the expression of a protein on the nucleic acid level include, but are not limited to, northern blotting, PCR, RT-PCR or real RT-PCR. PCR is well known in the art and is employed to make large numbers of copies of a target sequence. This is done on an automated cycler device, which can heat and cool containers with the reaction mixture in a very short time. The PCR, generally, consists of many repetitions of a cycle which consists of: (a) a denaturing step, which melts both strands of a DNA molecule and terminates all previous enzymatic reactions; (b) an annealing step, which is aimed at allowing the primers to anneal specifically to the melted strands of the DNA molecule; and (c) an extension step, which elongates the annealed primers by using the information provided by the template strand. Generally, PCR can be performed, for example, in a 50 μl reaction mixture containing 5 μl of 10× PCR buffer with 1.5 mM MgCl₂, 200 μM of each deoxynucleoside triphosphate, 0.5 μl of each primer (10 μM), about 10 to 100 ng of template DNA and 1 to 2.5 units of Taq polymerase. The primers for the amplification may be labeled or be unlabeled. DNA amplification can be performed, e.g., with a model 2400 thermal cycler (Applied Biosystems, Foster City, Calif.): 2 min at 94° C., followed by 30 to 40 cycles consisting of annealing (e. g. 30 s at 50° C.), extension (e. g. 1 min at 72° C., depending on the length of DNA template and the enzyme used), denaturing (e. g. 10 s at 94° C.) and a final annealing step, e.g. at 55° C. for 1 min as well as a final extension step, e.g. at 72° C. for 5 min. Suitable polymerases for use with a DNA template include, for example, E. coli DNA polymerase I or its Klenow fragment, T4 DNA polymerase, Tth polymerase, Taq polymerase, a heat-stable DNA polymerase isolated from Thermus aquaticus Vent, Amplitaq, Pfu and KOD, some of which may exhibit proof-reading function and/or different temperature optima. However, it is well known in the art how to optimize PCR conditions for the amplification of specific nucleic acid molecules with primers of different length and/or composition or to scale down or increase the volume of the reaction mix. The “reverse transcriptase polymerase chain reaction” (RT-PCR) is used when the nucleic acid to be amplified consists of RNA. The term “reverse transcriptase” refers to an enzyme that catalyzes the polymerization of deoxyribonucleoside triphosphates to form primer extension products that are complementary to a ribonucleic acid template. The enzyme initiates synthesis at the 3′-end of the primer and proceeds toward the 5′-end of the template until synthesis terminates. Examples of suitable polymerizing agents that convert the RNA target sequence into a complementary, copy-DNA (cDNA) sequence are avian myeloblastosis virus reverse transcriptase and Thermus thermophilus DNA polymerase, a thermostable DNA polymerase with reverse transcriptase activity marketed by Perkin Elmer. Typically, the genomic RNA/cDNA duplex template is heat denatured during the first denaturation step after the initial reverse transcription step leaving the DNA strand available as an amplification template. High-temperature RT provides greater primer specificity and improved efficiency. U.S. patent application Ser. No. 07/746, 121, filed Aug. 15, 1991, describes a “homogeneous RT-PCR” in which the same primers and polymerase suffice for both the reverse transcription and the PCR amplification steps, and the reaction conditions are optimized so that both reactions occur without a change of reagents. Thermus thermophilus DNA polymerase, a thermostable DNA polymerase that can function as a reverse transcriptase, can be used for all primer extension steps, regardless of template. Both processes can be done without having to open the tube to change or add reagents; only the temperature profile is adjusted between the first cycle (RNA template) and the rest of the amplification cycles (DNA template). The RT reaction can be performed, for example, in a 20 μl reaction mix containing: 4 μl of 5× AMV-RT buffer, 2 μl of oligo dT (100 μg/ml), 2 μl of 10 mM dNTPs, 1 μl total RNA, 10 Units of AMV reverse transcriptase, and H₂O to 20 μl final volume. The reaction may be, for example, performed by using the following conditions: The reaction is held at 70° C. for 15 minutes to allow for reverse transcription. The reaction temperature is then raised to 95° C. for 1 minute to denature the RNA-cDNA duplex. Next, the reaction temperature undergoes two cycles of 95° C. for 15 seconds and 60° C. for 20 seconds followed by 38 cycles of 90° C. for 15 seconds and 60° C. for 20 seconds. Finally, the reaction temperature is held at 60° C. for 4 minutes for the final extension step, cooled to 15° C., and held at that temperature until further processing of the amplified sample. Any of the above mentioned reaction conditions may be scaled up according to the needs of the particular case. The resulting products are loaded onto an agarose gel and band intensities are compared after staining the nucleic acid molecules with an intercalating dye such as ethidium bromide or SybrGreen. A lower band intensity of the sample treated with the antagonist as compared to a non-treated sample indicates that the antagonist successfully inhibits the expression of the protein.

Real-time PCR employs a specific probe, in the art also referred to as TaqMan probe, which has a reporter dye covalently attached at the 5′ end and a quencher at the 3′ end. After the TaqMan probe has been hybridized in the annealing step of the PCR reaction to the complementary site of the polynucleotide being amplified, the 5′ fluorophore is cleaved by the 5′ nuclease activity of Taq polymerase in the extension phase of the PCR reaction. This enhances the fluorescence of the 5′ donor, which was formerly quenched due to the close proximity to the 3′ acceptor in the TaqMan probe sequence. Thereby, the process of amplification can be monitored directly and in real time, which permits a significantly more precise determination of expression levels than conventional end-point PCR. Also of use in real time RT-PCR experiments is a DNA intercalating dye such as SybrGreen for monitoring the de novo synthesis of double stranded DNA molecules.

Methods for the determination of the expression of a protein on the amino acid level include, but are not limited to, western blotting or polyacrylamide gel electrophoresis in conjunction with protein staining techniques such as Coomassie Brilliant blue or silver-staining. The total protein is loaded onto a polyacrylamide gel and electrophoresed. Afterwards, the separated proteins are transferred onto a membrane, e.g. a polyvinyldifluoride (PVDF) membrane, by applying an electrical current. The proteins on the membrane are exposed to an antibody specifically recognizing the protein of interest. After washing, a second antibody specifically recognizing the first antibody and carrying a readout system such as a fluorescent dye is applied. The amount of the protein of interest is determined by comparing the fluorescence intensity of the protein derived from the sample with the fluorescence intensity of a control protein sample. In the case of assessing the potency of an antagonist or agonist, the amount of the protein of interest is determined by comparing the fluorescence intensity of the protein derived from the sample treated with the agonist or antagonist and the protein derived from a non-treated sample. A lower fluorescence intensity of the protein derived from the sample treated with the antagonist or a higher fluorescence intensity of the protein derived from the sample treated with the agonist indicates a functional antagonist or a functional agonist, respectively, of the protein. Also of use in protein quantification is the Agilent Bioanalyzer technique.

The above described methods for determining the expression level of a protein can also be used to assess the expression of UCP1 in brown and/or white adipocytes.

The increase of non-shivering thermogenesis in brown adipocytes in cell culture can be determined using established methods such as respirometry (Nedergaard J and Lindberg O. Norepinephrine-stimulated fatty-acid release and oxygen consumption in isolated hamster brown-fat cells. Influence of buffers, albumin, insulin and mitochondrial inhibitors. Eur J Biochem 1979; 95: 139-145), microcalorimetry (Tanaka E, et al. Regulation of heat production of brown adipocytes via typical and atypical beta-adrenoceptors in the rat. Jpn J Physiol. 1995; 45(6):1043-51), or thermography (Lee P, et al. Functional thermogenic beige adipogenesis is inducible in human neck fat. Int J Obes (Lond). 2014 February; 38(2):170-6). In vivo, brown adipose tissue thermogenesis can be assessed by indirect calorimetry (Böckler H, et al. Complete cold substitution of noradrenaline-induced thermogenesis in the Djungarian hamster, Phodopus sungorus. Experientia. 1982 Feb. 15; 38(2):261-2), infrared thermography (Crane J D, et al. A standardized infrared imaging technique that specifically detects UCP1-mediated thermogenesis in vivo. Mol Metab. 2014 Apr. 21; 3(4):490-4), and positron emission tomography with suitable tracers for glucose (18F-Deoxy-Glucose) and fatty acid (18F-fluoro-thiaheptadecanoic acid) uptake as well as overall metabolic rate (11C-acetate) (Ouellet V, et al. Brown adipose tissue oxidative metabolism contributes to energy expenditure during acute cold exposure in humans. J Clin Invest. 2012 February; 122(2):545-52).

Comparing these methods, at present the application of respirometry with cultured brown adipocytes is most effective as it requires a low number of cells and can be conducted in a multi-well format. In the art this method is regarded as the most reliable method to quantify UCP1-mediated thermogenesis. Preferably, the rate of non-shivering thermogenesis in cultured brown adipocytes in vitro is determined using an assay as developed in Li et al. (EMBO Rep. 2014 October; 15(10):1069-76) using oxygen consumption and extracellular acidification rate as read-out parameters. This setup has also been employed to determine the increase non-shivering thermogenesis in the example section.

As regards the prevention and/or treatment of dyslipidemia, the invention particularly encompasses the prevention and/or treatment of hyperlipidemias, preferably hypertriglyceridemia.

As regards the prevention and/or treatment of cachexia and without being bound by a scientific theory, the antagonist of the secretin receptor decreases non-shivering thermogenesis in brown adipocytes, thereby resulting in the prevention and/or treatment of cachexia. Antagonising the secretin receptor may be performed in combination treatment with IL-6 antagonists, nonsteroidal anti-inflammatory drugs (for example COX2 inhibitors) and/or beta3-adrenergic receptor antagonists.

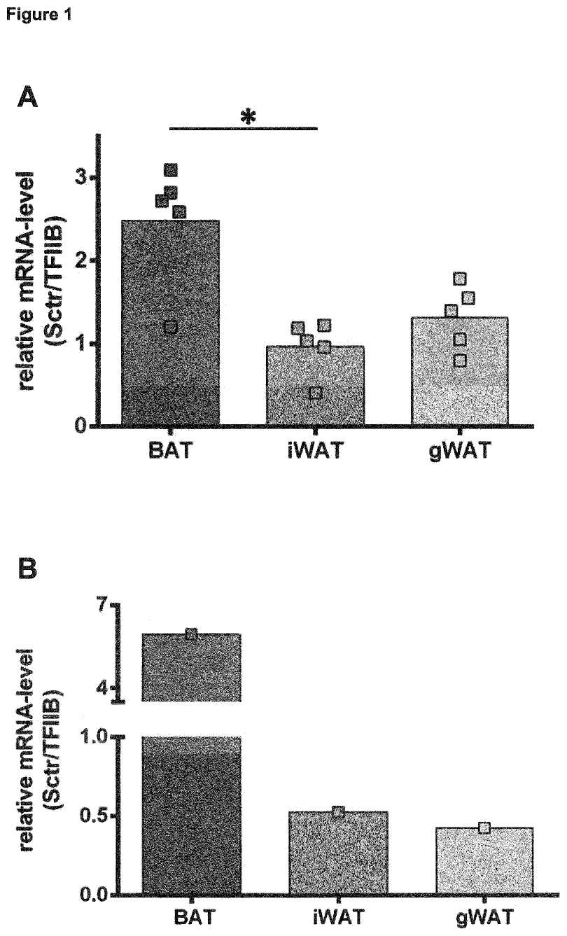

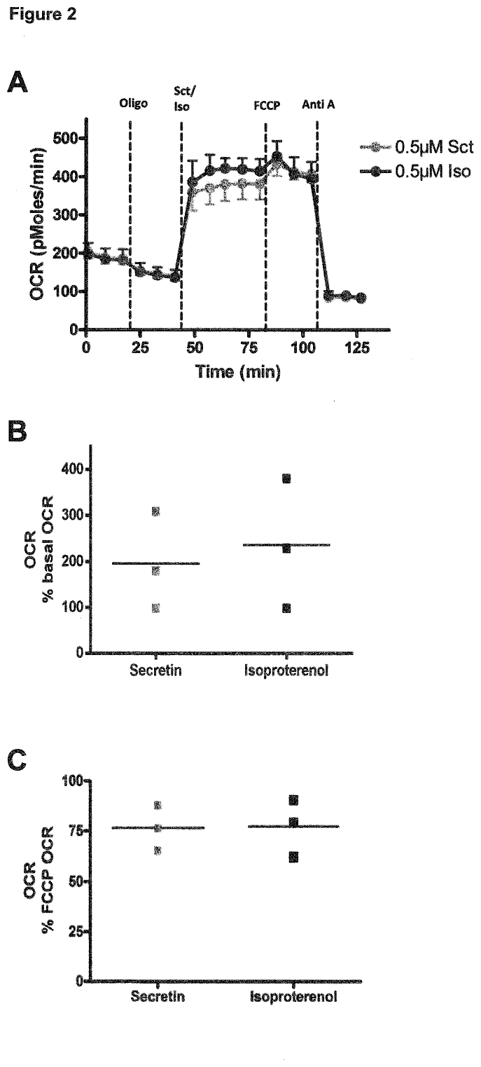

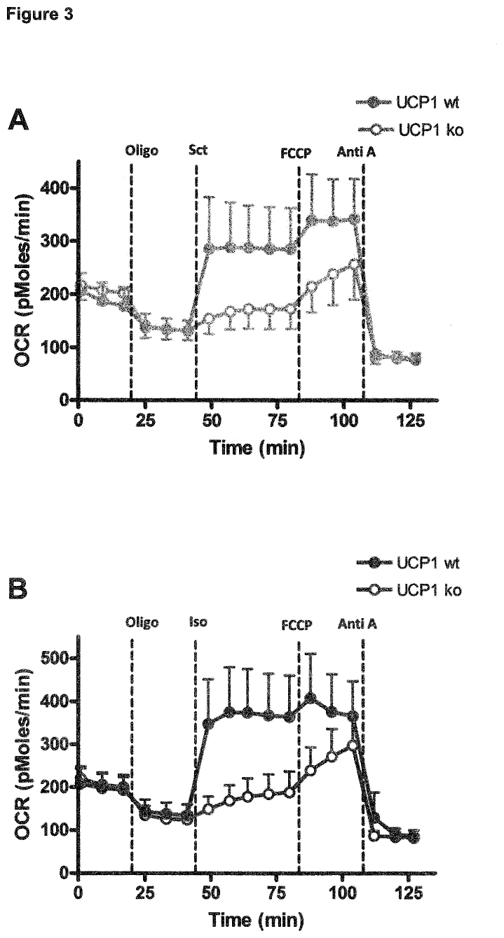

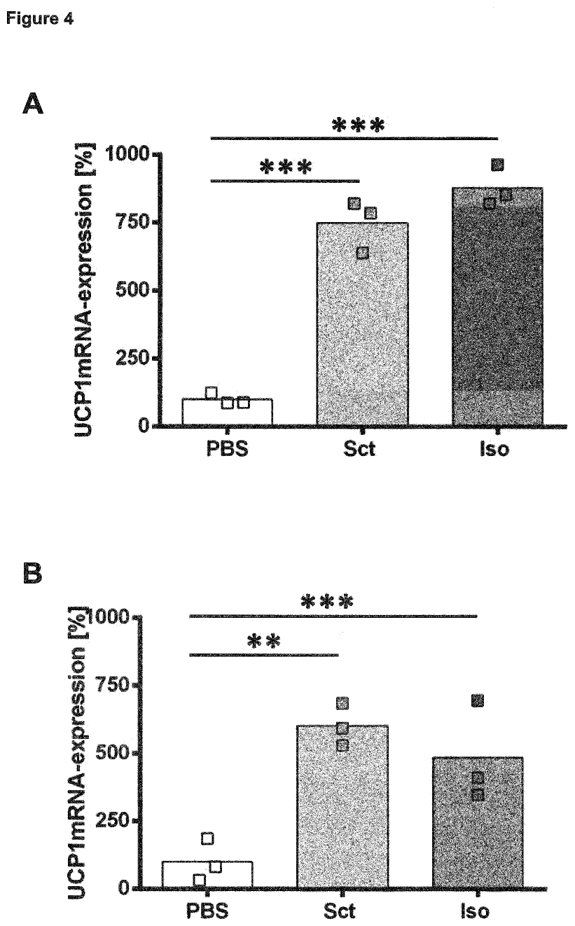

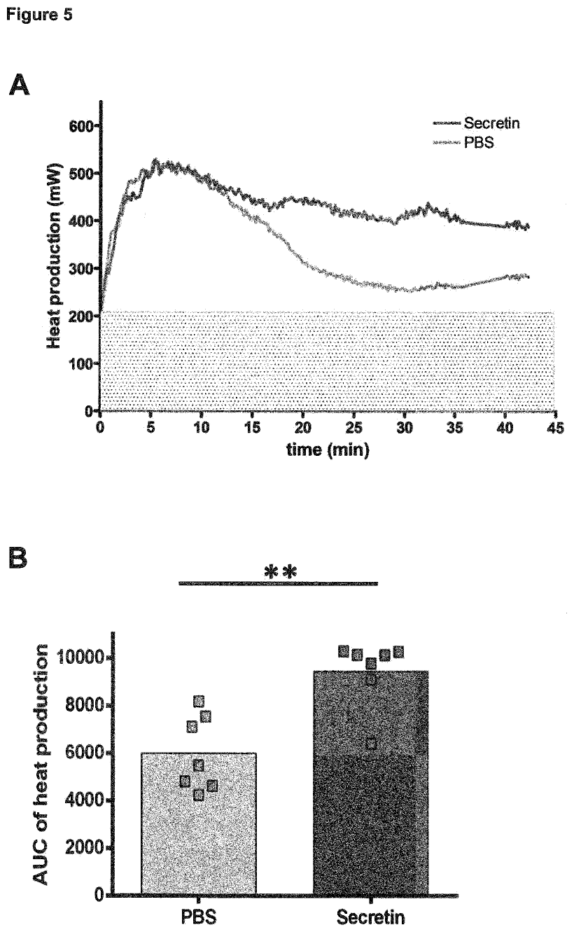

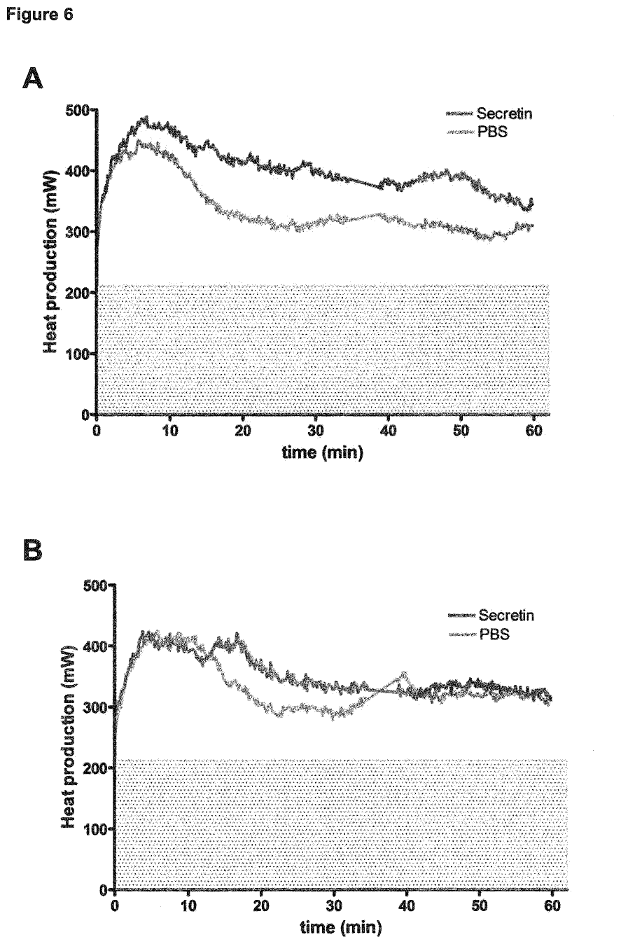

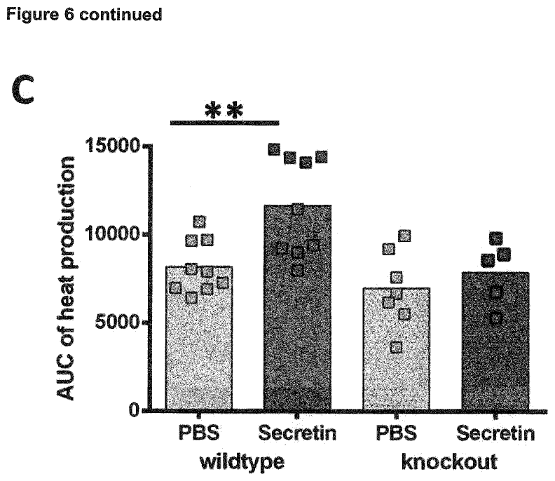

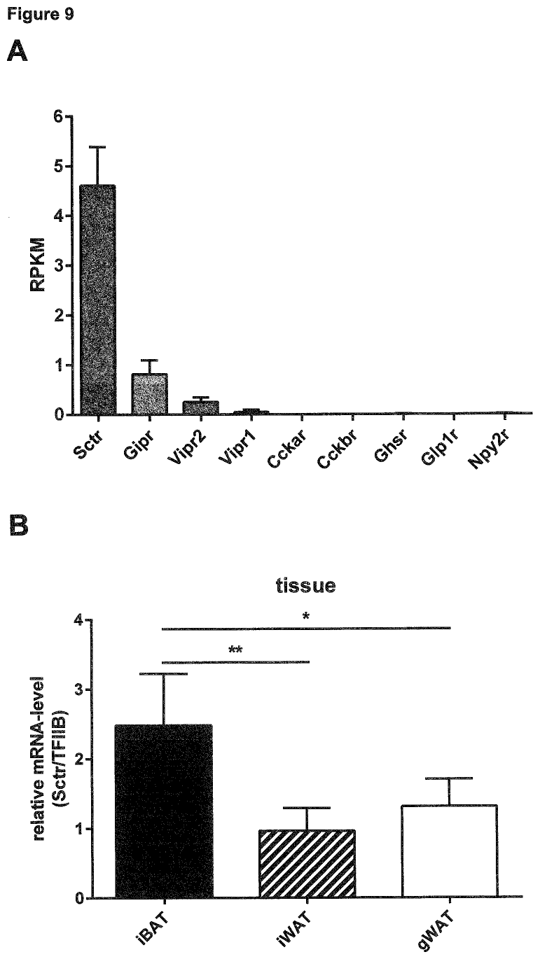

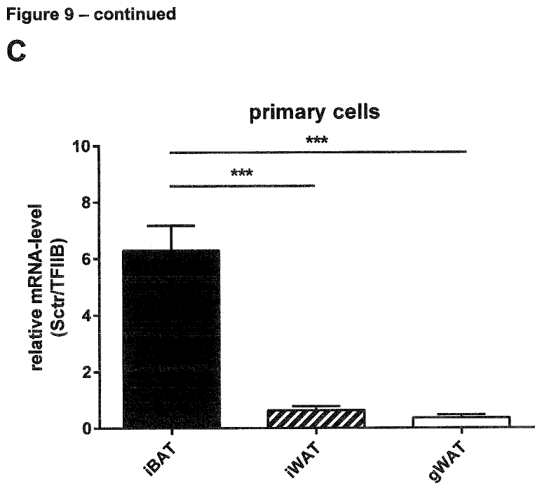

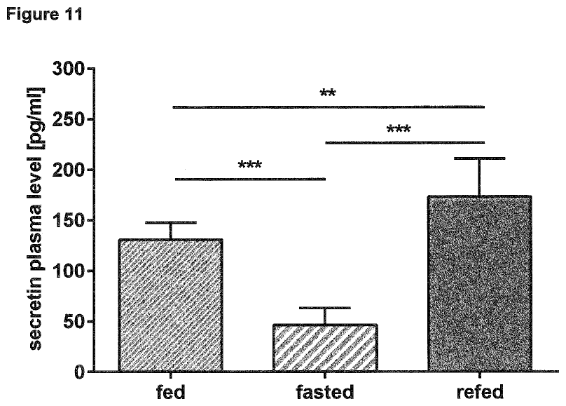

The invention is based inter alia on the results as included herein in the example section below and the accompanying figures. Specifically, it is shown that the secretin receptor is highly expressed in brown adipose tissue in mouse and is also present in white adipose tissue (see FIG. 1 and example 2; also FIG. 9A and example 9). Expression of the secretin receptor was also confirmed in primary adipocytes isolated from these tissues and was found to be much higher in primary adipocytes grown from brown adipose tissue (BAT) as compared to inguinal white adipose tissue (iWAT) and intra-abdominal gonadal white adipose tissue (gWAT) (FIG. 1B). Importantly, secretin significantly induces the oxygen consumption rate in brown adipocytes; in comparison to β-agonist isoproterenol, a known inducer of non-shivering thermogenesis via β-adrenergic action, secretin exhibited a similar activation profile of uncoupled respiration (see FIG. 2, and example 3) and could be shown to be dose-dependent with a higher potency at lower concentrations as compared to isoproterenol (data not shown). This effect is shown to be dependent on uncoupling protein 1 UCP1 as adipocytes from UCP1-knockout mice show no increase in oxygen consumption after stimulation with secretin as secretin receptor agonist (see FIG. 3, and example 4; see also FIG. 10A-C and example 10). Also, the thermogenic effect of secretin could not be blocked by the beta adrenergic receptor antagonist propanolol (see FIG. 10D and example 10). These effects could also be reproduced in vivo as evident from examples 6 and 7 and FIGS. 5 and 6 evidencing increased oxygen consumption after secretin injection into mice as well as increased heat production. Secretin also highly increased the expression of UCP1 in brown adipocytes (see FIG. 4 and example 5). Thus, besides the activating effects on non-shivering thermogenesis, secretin treatment of primary adipocytes differentiated in culture from the stromal vascular cell fraction of inguinal white adipose tissue (FIG. 4A) and interscapular brown adipose tissue (FIG. 4B) stimulates Ucp1 mRNA level to a level comparable with the beta-adrenergic receptor agonist isoproterenol. Thus, the agonistic stimulation of the secretin receptor also effects a recruitment of classical brown adipocytes and brite adipocytes, e.g. in inguinal white adipose tissue, (subsumed under the term “brown” adipocytes in accordance with the invention) via the increase in expression of UCP1. It could also be shown that the thermogenic effect can be inhibited, e.g. by secretin receptor knockdown using siRNA (see FIG. 10E and example 10) or upon treatment with a Protein Kinase A inhibitor to inhibit cAMP-PKA-signaling (see FIG. 1OF and example 11). It is further shown that secretin causes a significant inhibition of food intake in the initial phase of refeeding after fasting (see FIG. 7 and example 8) and that this inhibition depends on UCP1 (see FIG. 8 and example 8). It could further be shown that in response to overnight fasting, secretin levels were strongly reduced as compared to mice fed ad libitum, whereas refeeding mice after overnight fasting for 1 hour induced a strong increase in secretin levels (see FIG. 11 and example 11).

Taken together, it has been conclusively shown that agonistic stimulation of the secretin receptor increases UCP1-dependent non-shivering thermogenesis and therefore energy consumption in brown adipocytes and increases the expression of UCP1 in adipocytes from BAT as well as in adipocytes from WAT. Moreover, secretin was identified as a regulator in the control of energy expenditure and food intake. Taking this into account and considering the above described evidence of the role of WAT and BAT in energy homeostasis and the role of BAT in the diseases or disorders of energy homeostasis referred to in this embodiment and described herein further above, namely that it is accepted in the art that an increase in active BAT necessarily leads to an improvement in the primary manifestations of a disease or disorder of energy homeostasis like obesity, diabetes, dyslipidemia, insulin resistance, hyperglycemia, high blood pressure and/or metabolic syndrome, it can be plausibly expected that prevention and/or treatment of said disorders can be achieved. Furthermore, it is likely that secretin agonists will not exert detrimental cardiovascular side effects as observed with sympathomimetic drugs, e.g. beta3-adrenergic receptor agonists.

The definitions, explanations and preferred embodiments described herein above apply mutatis mutandis to all other embodiments of the invention described herein below unless explicitly stated otherwise.

In a preferred embodiment, the modulator of the secretin receptor is comprised in a pharmaceutical composition.

Preferably, the modulator, i.e. the antagonist or agonist, in this embodiment of the present invention is comprised in a pharmaceutical composition, optionally further comprising a pharmaceutically acceptable carrier, excipient and/or diluents. The term “pharmaceutical composition”, as used herein, relates to a composition for administration to a patient, preferably a human patient. The pharmaceutical composition of the invention comprises at least one, such as at least two, e.g. at least three, in further embodiments at least four such as at last five of the above mentioned antagonists or agonists. The invention also envisages mixtures of different antagonists or mixtures of different agonists of the secretin receptor falling into one or more of the different antagonist or agonist classes defined herein above. In cases where more than one antagonist or agonist is comprised in the pharmaceutical composition, it is understood that none of these antagonists or agonists has any essentially inhibitory effect on the other antagonists or agonists also comprised in the composition. This does not exclude the possibility that synergistic effects may be observed.

The composition may be in solid, liquid or gaseous form and may be, inter alia, in a form of (a) powder(s), (a) tablet(s), (a) solution(s) or (an) aerosol(s).

It is preferred that said pharmaceutical composition comprises a pharmaceutically acceptable carrier, excipient and/or diluent. Examples of suitable pharmaceutical carriers, excipients and/or diluents are well known in the art and include phosphate buffered saline solutions, water, emulsions, such as oil/water emulsions, various types of wetting agents, sterile solutions etc. Compositions comprising such carriers can be formulated by well known conventional methods. These pharmaceutical compositions can be administered to the subject at a suitable dose. Administration of the suitable compositions may be effected by different ways, e.g., by intravenous, intraperitoneal, subcutaneous, intramuscular, topical, intradermal, intranasal or intrabronchial administration. It is particularly preferred that said administration is carried out by delivery, e.g., to a site in the bloodstream such as a coronary artery or directly into the respective tissue, e.g., by injection. The compositions of the invention may also be administered directly to the target site, e.g., by biolistic delivery to an external or internal target site, like brown adipose tissue. The dosage regimen will be determined by the attending physician and clinical factors. As is well known in the medical arts, dosages for any one patient depend upon many factors, including the patient's size, body surface area, age, the particular compound to be administered, sex, time and route of administration, general health, and other drugs being administered concurrently. Proteinaceous pharmaceutically active matter may be present in amounts between 1 ng and 10 mg/kg body weight per dose; however, doses below or above this exemplary range are envisioned, especially considering the aforementioned factors. If the regimen is a continuous infusion, it should also be in the range of 0.01 μg to 10 mg units per kilogram of body weight per minute. The continuous infusion regimen may be completed with a loading dose in the dose range of 1 ng and 10 mg/kg body weight.

Progress can be monitored by periodic assessment. The compositions of the invention may be administered locally or systemically. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Conventional excipients include binding agents, fillers, lubricants and wetting agents.

Also, the invention relates to a method of treating a disease or disorder of energy homeostasis by administering a modulator of the secretin receptor to a subject in need thereof, wherein (a) said modulator is a secretin receptor agonist and said disease or disorder is obesity, dyslipidemia, diabetes, insulin resistance, hyperglycemia, high blood pressure or metabolic syndrome, whereby the secretin receptor agonist increases non-shivering thermogenesis in brown adipocytes and/or increases the expression of uncoupling protein 1 (UCP1) in brown adipocytes resulting in the prevention and/or treatment of said disease or disorder; or (b) wherein said modulator is a secretin receptor antagonist and said disease or disorder is cachexia. All definitions given herein, in particular above, also apply mutatis mutandis to this embodiment.

In a further preferred embodiment, the modulator of the secretin receptor is to be co-administered with at least one other pharmaceutically active agent.

It is preferably also envisaged that said pharmaceutical composition comprises at least one other/further agent known in the art to be effective in the treatment of the above mentioned diseases, which is not a secretin receptor agonist or antagonist. Since the pharmaceutical preparation of the present invention relies on the above mentioned antagonists or agonists of the secretin receptor, it is preferred that the mentioned at least one further agent is only used as a supplement, i.e. at a reduced dose as compared to the recommended dose when used as the only drug, so as to e.g. reduce side effects conferred by said further agent. Of course, also pharmaceutically active agents that are known to be not effective in the treatment of the above mentioned disease can be administered together with the modulator of the secretin receptor.

In more preferred embodiment and in the case of the modulator of the secretin receptor being a secretin receptor agonist said at least one other pharmaceutically active agent is selected from the group consisting of direct or indirect sympathomimetics, atrial natriuretic peptide and ANP/BNP receptor agonists.

Said direct sympathomimetics are selected, e.g., from the group consisting of noradrenalin, isoproterenol, BRL 35135 (murine beta3-AR agonist), ICI D7114 (murine beta3-AR agonist), CGP-12177A (murine beta3-AR agonist), CL 316243 (murine beta3-AR agonist), mirabegron (2-(2-Amino-1,3-thiazol-4-yl)-N-[4-(2-{[(2R)-2-hydroxy-2-phenylethyl]amino}ethyl)phenyl]acetamide), and solabegron (3′-[(2-{[(2R)-2-(3-Chlorophenyl)-2-hydroxyethyl]amino}ethyl)amino]-3-biphenylcarboxylic acid; the latter two compounds are both examples of agonists specific for human beta3-AR); said indirect sympathomimetics can be, e.g. ephedrine or methylphenidate; said atrial natriuretic peptide can be, e.g. ANP or BNP; and an ANP/BNP receptor agonist can be, e.g., AP-811. Said noradrenalin may be native noradrenalin. Said beta-adrenergic agonist may be a beta3-adrenergic agonist and/or said beta3-adrenergic agonist may be CL 316243.

In another embodiment the invention relates to a method of increasing non-shivering thermogenesis in brown adipocytes and/or increasing the expression of uncoupling protein 1 (UCP1) in brown adipocytes, comprising the step of contacting brown adipocytes with a secretin receptor agonist, thereby increasing non-shivering thermogenesis in brown adipocytes and/or increasing the expression of uncoupling protein 1 (UCP1) in brown adipocytes.

The definitions, explanations and preferred embodiments provided herein above also apply mutatis mutandis to this embodiment.

The terms “brown adipocytes” and “white adipocytes” as used herein can refer to a single and/or isolated adipocyte or, preferably, to several adipocytes such as, e.g., an adipose tissue or cell culture thereof or an adipocytes cell culture. Accordingly, these adipocytes may be part of a multicellular entity such as a (isolated) tissue, an organism or a cell culture. In other words, the method can be performed in vivo or, preferably, ex vivo or in vitro. When performed in vivo, the method preferably excludes methods of treatment of the human or animal body or diagnostic methods practised on the human or animal body.

Adipocytes to be used may originate from established cell lines but may also include cell of a primary cell line established from a tissue sample. Preferably, the adipocytes used originate from the same cell line or are established from the same tissue. Suitable brown and white adipocytes cell lines may also be purchased from a number of suppliers such as, for example, the American tissue culture collection (ATCC), the German Collection of Microorganisms and Cell Cultures (DSMZ). Exemplary brown adipocytes cell lines are immortalized stromal vascular fractions isolated from brown adipose tissue (such as WT-1 cells) and HIB-1B cell line; exemplary white adipocyte cell lines are 3T3L-1 cells. As outlined herein above in another context, the method is, preferably, performed with adipocytes that can originate from mammals such as, e.g., mice, cats or dogs and, more preferably, from humans.

The agonist of the secretin receptor is contacted with the adipocytes under conditions that allow said agonist to exert its effects on the adipocytes. These conditions depend on the agonists used and are adapted accordingly.

In a further embodiment, the invention relates to a method of decreasing non-shivering thermogenesis in brown adipocytes comprising the step of contacting brown adipocytes with a secretin receptor antagonist, thereby decreasing non-shivering thermogenesis in brown adipocytes.

The definitions, explanations and preferred embodiments provided herein above, in particular those relating to the method of increasing non-shivering thermogenesis in brown adipocytes and/or increasing the expression of uncoupling protein 1 (UCP1) in brown adipocytes, also apply mutatis mutandis to this embodiment.

In an even further embodiment, the invention relates to a method of identifying a secretin receptor agonist capable of increasing non-shivering thermogenesis in brown adipocytes and/or increasing the expression of uncoupling protein 1 (UCP1) in brown adipocytes, comprising the steps of: (a) determining the level of non-shivering thermogenesis in brown adipocytes and/or the expression level of uncoupling protein 1 (UCP1) in brown adipocytes or obtaining established standard values of the level of non-shivering thermogenesis and/or the of the expression of uncoupling protein 1 (UCP1); (b) contacting said adipocytes of step (a) or other brown adipocytes with a test compound; (c) determining the level of non-shivering thermogenesis in said brown adipocytes of step (b) and/or the expression level of uncoupling protein 1 (UCP1) in said brown adipocytes of step (b) after contacting with the test compound; and (d) comparing the level of non-shivering thermogenesis in said brown adipocytes and/or the expression level of uncoupling protein 1 (UCP1) in said brown adipocytes determined in step (c) with the level of non-shivering thermogenesis in said brown adipocytes and/or the expression level of uncoupling protein 1 (UCP1) in said brown adipocytes determined in step (a) or to standard values of the level of the level of non-shivering thermogenesis and/or the of the expression of uncoupling protein 1 (UCP1) obtained in step (a), wherein an increase in the level of non-shivering thermogenesis in said brown adipocytes determined in step (c) as compared to said level determined or obtained in step (a) and/or an increase in the expression level of uncoupling protein 1 (UCP1) in said brown adipocytes determined in step (c) as compared to said level determined or obtained in step (a) indicates that said test compound is a secretin receptor agonist capable of increasing non-shivering thermogenesis in brown adipocytes and/or increasing the expression of uncoupling protein 1 (UCP1) in brown adipocytes.

This embodiment relates to a cellular screen, wherein agonists of the secretin receptor may be identified which exert their activity by physically interacting with the secretin receptor to activate the latter, or alternatively (or additionally) by functionally interacting with the secretin receptor, i.e., by interfering with the pathway(s) present in the cells employed in the cellular assay, preferably upstream of the secretin receptor, to activate said secretin receptor. Furthermore, such compounds may, as described above, alter the affinity or rate of binding of a known ligand, such as secretin, to the receptor or compete with a ligand for binding to the receptor or displace a ligand bound to the receptor. As a result, one or more of the specific biological functions or activities of the secretin receptor in accordance with the invention (increase of non-shivering thermogenesis and/or increase in the expression level of UCP1) are modified either directly or indirectly, which can be measured as an increased level of one or more of said specific biological functions or activities mentioned.

Preferably, the method of the invention covered in this embodiment is a method not encompassing methods for treatment of the human or animal body by surgery or therapy. More preferred, the method is an ex vivo or in vitro method, i.e. carried out ex vivo or in vitro. In vitro methods offer the possibility of establishing high-throughput assays, as described above.

The term “said adipocytes of step (a) or other brown adipocytes” as used herein in step (b) of the method refers either i) to the adipocytes used in step (a) or ii) to other respective brown adipocytes of the same origin as the adipocytes of step (a), so as to allow a reasonable comparability of the results as laid out in step (d) when not the same set of adipocytes as in step (a) is used. The same limitations and definitions with regard to the term “adipocytes” given herein above apply also to this embodiment mutatis mutandis.

In accordance with the method of the invention covered in this embodiment, the determination step (a) alternatively also encompasses the obtainment of established standard values of the level of non-shivering thermogenesis in brown adipocytes and/or of the expression level of uncoupling protein 1 (UCP1) in brown adipocytes. As such, step (d) of this alternative encompasses the comparison to said established standard values.

“Established standard values” are values that have previously been generated in the respective cells used in the assay and are at the disposal of the person implementing the method of the invention. A corresponding setup may prove beneficial in particular in view of high throughput screenings (HTS), since the necessity of actually experimentally determining the corresponding levels according to step (a) for each assay does not arise. As described hereinabove, the secretin receptor plays a heretofore unknown key role in energy metabolism. Therefore, the use of the secretin receptor as a target for the identification of agonists of the latter as defined herein, which may be suitable as a lead compound and/or for the prevention and/or treatment of the above specified diseases and disorders, is also encompassed by the present invention. It is envisaged that an increase in non-shivering thermogenesis in brown adipocytes and/or an increase in the expression level of UCP1 in brown adipocytes conferred by a test compound acting as agonist as described above will contribute to the prevention and/or treatment of the diseases and disorders referred to herein above. Accordingly, measurement of an increase in non-shivering thermogenesis in brown adipocytes and/or an increase in the expression level of UCP1 in brown adipocytes is suitable as a readout of the above-described assay. Accordingly, an agonist identified according to the method may be suitable as a lead compound and/or for the prevention and/or treatment of the above specified diseases and disorders.

For example, the above-mentioned brown adipocytes may exhibit a certain rate of non-shivering thermogenesis before contact with the test compound and said rate may be increased after contacting the brown adipocytes with the test compound, indicating that an agonist has been identified. Preferably, non-shivering thermogenesis and/or the expression level of UCP1 is increased by, for example, at least 10, at least 20, at least 30, at least 40 or at least 50% after contacting the cell with the test compound as compared to the determined values of the same readout parameters before contacting the respective adipocytes with the test compound. More preferred, non-shivering thermogenesis and/or the expression level of UCP1 after contacting the cell with the test compound is increased by, for example, at least 60, at least 70, at least 80, at least 90 or at least 95% as compared to the same readout parameters before contacting the cell with the test compound. Higher increases are also envisaged. As such, non-shivering thermogenesis and/or the expression level of UCP1 after contacting the cell with the test compound is increased by 100%, preferably more than 100% such as 150%, 200%, 500% or 1000% as compared to the same readout parameters before contacting the cell with said test compound. The term “non-shivering thermogenesis and/or the expression level of UCP is changed by (at least) . . . %” refers to a relative alteration compared to the non-shivering thermogenesis and/or the expression level of UCP before contacting the respective adipocytes with the test compound. For example and referring to absolute read-out parameter values, an increase of at least 50% means that after contacting the adipocytes with the test compound the non-shivering thermogenesis and/or the expression level of UCP1 has the read-out parameter value of 15 or more as compared to non-shivering thermogenesis and/or the expression level of UCP1 before contacting the cell with the test compound, where the read-out parameter value was, thus, 10.

It is understood that the increase in non-shivering thermogenesis and/or the increase in UCP1 expression level that may be determined according to the method of the invention is associated with an agonistic action of the designated secretin receptor agonist on the secretin receptor. In order to determine that the observed increase in non-shivering thermogenesis and/or increase in the expression level of UCP1 results from the agonistic effects of the designated secretin receptor agonist (i.e. the test(ed) compound) on the secretin receptor as defined herein above, one can incorporate further experimental steps for determining whether the test compound actually exerts said effect on the secretin receptor. Accordingly, the method of the invention can, preferably, comprise a further step (e) of determining prior to, simultaneously with or after any one of the preceding steps (a) to (d) whether said test agent modulates the secretin receptor. As described herein above in relation to agonists of the secretin receptor, there exist various ways an agonist can modulate the secretin receptor in an agonistic manner such as affecting secretin receptor protein translation as well as secretin receptor gene transcription or direct and/or indirect interaction with the secretin receptor protein. Accordingly, said step (e) of whether the test compound modulates the secretin receptor can be performed at any stage of the method according to the invention. In some instances it may be worthwhile to carry out step (e) as an initial step to narrow down the test compounds only to those that modulate secretin in an agonistic manner before testing them for their capability to increase non-shivering thermogenesis in brown adipocytes and/or increase the expression level of UCP1 in brown adipocytes.

Hence, step (e) may involve a step (1) where it is determined whether the test compound binds to the secretin receptor or an activator of the secretin receptor. The term “cellular activator of the secretin receptor” relates to an entity that is endogenously present in the respective adipocyte and is naturally involved in the recruitment of the secretin receptor. As described herein above various methods exist to assess the binding of two molecular entities to each other.

As an alternative, or additionally, step (e) may involve steps (2)(i) to (2)(iii) where it is determined whether the test compound modulates the secretin receptor by altering the levels of the secretin receptor protein or the secretin receptor transcript. For example, the adipocytes exhibit a detectable level of secretin receptor protein or secretin receptor transcript before contacting with the test compound and the level of secretin receptor protein or secretin receptor transcript is higher after contacting the adipocytes with the test compound, indicating that the test compound modulates the secretin receptor by increasing it quantitatively and that this may affect the biological functions or activities of the secretin receptor in the sense of an agonist as defined herein above. On the other hand, a decrease in the level of secretin receptor protein or secretin receptor transcript after contacting the adipocytes with the test compound indicates that the test compound modulates the secretin receptor by decreasing it quantitatively in the sense of an antagonist as defined herein above and that this may affect the biological function or activity of the secretin receptor by decreasing non-shivering thermogenesis. Measurements of protein levels as well as of transcript level can be accomplished in several ways, as described above.

In a further preferred embodiment of the method of the invention, the step (e) comprises: (1) assessing whether said test compound binds to the secretin receptor or a cellular activator of the secretin receptor, wherein binding of said test compound to said secretin receptor or said cellular activator of the secretin receptor indicates that said test compound modulates the secretin receptor in an agonistic manner; and/or (2)(i) determining the level of secretin receptor protein or secretin receptor transcript in adipocytes according to step (a) of the method of identifying an agonist of the secretin receptor described herein above; (2)(ii) determining the level of secretin receptor protein or secretin receptor transcript in said cell according to step (b) of the method of identifying described herein above after contact with said test compound; and (2)(iii) comparing the level of secretin receptor protein or secretin receptor transcript determined in step (2)(ii) with the secretin receptor protein or secretin receptor transcript level determined in step (2)(i), wherein an increase of secretin receptor protein or secretin receptor transcript level in step (2)(ii) as compared to step (2)(i) indicates that the test compound can modulate the secretin receptor in agonistic manner.

As outlined herein above, it is understood that the test compound actually modulates the secretin receptor in an agonistic manner as defined herein above so that the increased biological functions or activities of the secretin receptor detected in the course of executing the method of the invention are related to the secretin receptor.

Preferably, for all embodiments described herein, the test compounds or the modulators of the secretin receptor, in particular an agonist of the secretin receptor, are active site binders on the secretin receptor or do not bind directly to the target, but still influence its biological functions or activities, for example, by binding to and/or activating the function of or increasing the expression of upstream cellular molecules that are members of a pathway which comprises the target, i.e. the secretin receptor. Thus, modulators indirectly modulating the secretin receptor are interacting with cellular molecules upstream of the secretin receptor. In other words, and in relation to agonists of the secretin receptor, the latter bind preferably to the secretin receptor or interact with cellular molecules upstream of the secretin receptor and thereby result in the increase of non-shivering thermogenesis in brown adipocytes and/or in an increase of the expression of uncoupling protein 1 (UCP1) in brown adipocytes.

It is understood in accordance with the invention that a receptor agonist capable of increasing non-shivering thermogenesis in brown adipocytes and/or increasing the expression of uncoupling protein 1 (UCP1) in brown adipocytes identified according to the screening method of the invention also has the activity of decreasing food intake in a UCP1-dependent manner. If need be said activity can be assessed, e.g., by using a method as disclosed in example 8. Alternatively, a method of identifying a secretin receptor agonist capable of decreasing food intake in UCP1-dependent manner is disclosed herein, comprising the steps of: (a) determining the level of food intake in a subject; (b) contacting said subject of step (a) or another subject with a test compound; (c) determining the level of food intake in said subject of step (b) after contacting with the test compound; and (d) comparing the level of food intake in said subject determined in step (c) with the level of food intake in said subject determined in step (a), wherein a decrease in the level of food intake in said subject determined in step (c) as compared to said level determined in step (a) indicates that said test compound is a secretin receptor agonist capable of decreasing food intake in UCP1-dependent manner.

In relation to the method of identifying a secretin receptor agonist capable of decreasing food intake in UCP1-dependent manner, the term a “subject” preferably does not relate to a human subject, but preferably to rodents, such as, for example, mice or rats. Also preferred is that said method does not encompass methods for treatment of the human or animal body by surgery or therapy.

Additionally, the invention also relates to a method of identifying a secretin receptor antagonist capable of decreasing non-shivering thermogenesis in brown adipocytes, comprising the steps of: (a) determining the level of non-shivering thermogenesis in brown adipocytes; (b) contacting said adipocytes of step (a) or other brown adipocytes with a test compound; (c) determining the level of non-shivering thermogenesis in said brown adipocytes of step (b) after contacting with the test compound; and (d) comparing the level of non-shivering thermogenesis in said brown adipocytes determined in step (c) with the level of non-shivering thermogenesis in said brown adipocytes determined in step (a), wherein an decrease in the level of non-shivering thermogenesis in said brown adipocytes determined in step (c) as compared to said level determined in step (a) indicates that said test compound is a secretin receptor antagonist capable of decreasing non-shivering thermogenesis in brown adipocytes.

The definitions, explanations and preferred embodiments outlined herein, above, for the method of identifying agonists of the secretin receptor also apply mutatis mutandis to this embodiment.

In a preferred embodiment, the methods of identifying an agonist or antagonist of the secretin receptor described herein above, comprise a further step of determining prior to, simultaneously with or after any one of the preceding steps (a) to (d) whether said test compound modulates the secretin receptor.

It is understood that the increase in non-shivering thermogenesis and/or UCP1 expression level that may be determined according to the method of the invention is associated with an agonistic action on the secretin receptor following from the designation secretin receptor agonist, whereas the decrease in non-shivering thermogenesis that may be determined according to the method of the invention is associated with an antagonistic action on the secretin receptor following from the designation secretin receptor antagonist. In order to determine that the observed increase in non-shivering thermogenesis and/or the expression level of UCP1 results from the agonistic effects on the secretin receptor as defined herein above of the test compound, one can determine whether the test compound actually exerts said effect on the secretin receptor. Accordingly, the method of the invention can comprise a further step (e) of determining prior to, simultaneously with or after any one of the preceding steps (a) to (d) whether said test agent modulates the secretin receptor. As described herein above in relation to agonists of the secretin receptor, there exist various ways an agonist can modulate the secretin receptor in an agonistic manner such as affecting secretin receptor protein translation as well as secretin receptor gene transcription or direct and/or indirect interaction with the secretin receptor protein. Accordingly, said step (e) of whether the test compound modulates the secretin receptor can be performed at any stage of the method according to the invention. In some instances it may be worthwhile to carry out step (e) as initial step to narrow down the test compounds only to those that modulate secretin in an agonistic manner before testing them for their capability to increase non-shivering thermogenesis in brown adipocytes and/or increase the expression level of UCP1 in brown adipocytes.

Hence, step (e) may involve a step (1) where it is determined whether the test compound binds to the secretin receptor or an activator of the secretin receptor. The term “cellular activator of the secretin receptor” relates to an entity that is endogenously present in the respective adipocyte and is naturally involved in the recruitment of the secretin receptor. As described herein above various methods exist to assess the binding of two molecular entities to each other.

As an alternative, or additionally, step (e) may involve steps (2)(i) to (2)(iii) where it is determined whether the test compound modulates the secretin receptor by altering the levels of the secretin receptor protein or the secretin receptor transcript. For example, the adipocytes exhibit a detectable level of secretin receptor protein or secretin receptor transcript before contacting with the test compound and the level of secretin receptor protein or secretin receptor transcript is higher after contacting the adipocytes with the test compound, indicating that the test compound modulates the secretin receptor by increasing it quantitatively and that this may affect the biological functions or activities of the secretin receptor in the sense of an agonist as defined herein above. On the other hand, a decrease in the level of secretin receptor protein or secretin receptor transcript after contacting the adipocytes with the test compound indicates that the test compound modulates the secretin receptor by decreasing it quantitatively in the sense of an antagonist as defined herein above and that this may affect the biological function or activity of the secretin receptor by decreasing non-shivering thermogenesis. Measurements of protein levels as well as of transcript level can be accomplished in several ways, as described above.

In a further preferred embodiment of the method of the invention, the step (e) comprises: (1) assessing whether said test compound binds to the secretin receptor or a cellular activator of the secretin receptor, wherein binding of said test compound to said secretin receptor or said cellular activator of the secretin receptor indicates that said test compound modulates the secretin receptor in an agonistic or antagonistic manner; and/or (2)(i) determining the level of secretin receptor protein or secretin receptor transcript in adipocytes according to step (a) of the method of identifying an agonist or an antagonist of the secretin receptor described herein above; (2)(ii) determining the level of secretin receptor protein or secretin receptor transcript in said cell according to step (b) of the method of identifying described herein above after contact with said test compound; and (2)(iii) comparing the level of secretin receptor protein or secretin receptor transcript determined in step (2)(ii) with the secretin receptor protein or secretin receptor transcript level determined in step (2)(i), wherein an increase of secretin receptor protein or secretin receptor transcript level in step (2)(ii) as compared to step (2)(i) indicates that the test compound can modulate the secretin receptor in agonistic manner, whereas a decrease of secretin receptor protein or secretin receptor transcript level in step (2)(ii) as compared to step (2)(i) indicates that the test compound can modulate the secretin receptor in an antagonistic manner.

As outlined herein above, it is understood that the test compound actually modulates the secretin receptor in an agonistic or antagonistic manner as defined herein above so that the increased or decreased biological functions or activities of the secretin receptor detected in the course of executing the method of the invention are related to the secretin receptor.

Preferably, for all embodiments described herein, the test compound or the modulator of the secretin receptor, in particular an agonist of the secretin receptor, are active site binders on the secretin receptor or do not bind directly to the target, but still influence its biological functions or activities, for example, by binding to and/or activating/deactivating the function of or increasing/decreasing the expression of upstream cellular molecules that are members of a pathway which comprises the target, i.e. the secretin receptor. Thus, modulators indirectly modulating the secretin receptor are interacting with cellular molecules upstream of the secretin receptor. In other words, and in relation to agonists of the secretin receptor, the latter bind preferably to the secretin receptor or interact with cellular molecules upstream of the secretin receptor and thereby result in the increase of non-shivering thermogenesis in brown adipocytes and/or in an increase of the expression of uncoupling protein 1 (UCP1) in brown adipocytes; in relation to antagonists of the secretin receptor, the latter bind preferably to the secretin receptor or interact with cellular molecules upstream of the secretin receptor and thereby result in the decrease of non-shivering thermogenesis in brown adipocytes. Most preferred is that an agonist or antagonist of the secretin receptor binds to the secretin receptor.

In another embodiment, the invention relates to the use of the secretin receptor for screening (a) for secretin receptor agonists that increase non-shivering thermogenesis in brown adipocytes and/or increase the expression of uncoupling protein 1 (UCP1) in brown adipocytes and/or decrease food intake in a UCP1-dependent manner; and/or (b) for secretin receptor antagonists that decrease non-shivering thermogenesis in brown adipocytes.

The use of the secretin receptor for screening for agonists and antagonists of the secretin receptor according to the invention has been exemplified by the methods for identifying said agonists and antagonists herein above. Thus, the definitions, explanations and preferred embodiments also apply to this embodiment mutatis mutandis.

In a further embodiment, the invention relates to the use of the secretin receptor agonist to increase non-shivering thermogenesis in brown adipocytes and/or to increase the expression of uncoupling protein 1 (UCP1) in brown adipocytes and/or to decrease food intake in a UCP1-dependent manner for reducing body weight for cosmetic purposes.

While the invention concerns embodiments that relate to the prevention and/or treatment of diseases or disorders of energy homeostasis, where the treatment and/or prevention is associated with a reduction of body weight due to the action of agonists of the secretin receptor, there are cases where subject desires a reduction of weight for cosmetic purposes. It is understood that in these cases, there is no medical necessity of a medical necessity for reducing body weight. “Medical necessity” is meant to refer to the case where the loss of body weight is determined by a medical professional to be necessary to treat an existing disease and/or to prevent a disease from developing. The disease is, preferably, a disease as outlined herein above. This is adequately expressed by the terms “for cosmetic purposes” used herein. In other words, the use is a non-therapeutic use.

Also for this embodiment, the definitions, explanations and preferred embodiments given herein above also apply to this embodiment mutatis mutandis.

Further, the invention relates in an additional embodiment to the use of the secretin receptor antagonist to decrease thermogenesis in brown adipocytes for increasing body weight for cosmetic purposes.

Also for this embodiment, the definitions, explanations and preferred embodiments given herein above, in particular in the embodiment relating to the use of the secretin receptor agonist to increase non-shivering thermogenesis in brown adipocytes and/or increase the expression of uncoupling protein 1 (UCP1) in brown adipocytes for reducing body weight for cosmetic purposes, also apply to this embodiment mutatis mutandis.

Conceivably, there may be cases where a subject would like to increase body weight for cosmetic purposes. For example, in certain cultures a more corpulent appearance is desirable and with the use described herein said appearance may be attained.

In a preferred embodiment of the different embodiments of the invention, the agonist of the secretin receptor is selected from the group consisting of a small molecule, an antibody or a fragment or derivative thereof, an antibody mimetic, an aptamer, an siRNA, an shRNA, a miRNA, a ribozyme, an antisense nucleic acid molecule, secretin or a fragment thereof, a secretin analogue, or a stimulant of secretin release.

A “small molecule” as used herein may be, for example, an organic molecule. Organic molecules relate or belong to the class of chemical compounds having a carbon basis, the carbon atoms linked together by carbon-carbon bonds. The original definition of the term organic related to the source of chemical compounds, with organic compounds being those carbon-containing compounds obtained from plant or animal or microbial sources, whereas inorganic compounds were obtained from mineral sources. Organic compounds can be natural or synthetic. Alternatively, the “small molecule” in accordance with the present invention may be an inorganic compound. Inorganic compounds are derived from mineral sources and include all compounds without carbon atoms (except carbon dioxide, carbon monoxide and carbonates). Preferably, the small molecule has a molecular weight of less than about 2000 amu, or less than about 1000 amu such as less than about 500 amu, and even more preferably less than about 250 amu. The size of a small molecule can be determined by methods well-known in the art, e.g., mass spectrometry. The small molecules may be designed, for example, based on the crystal structure of the target molecule, where sites presumably responsible for the biological activity, can be identified and verified in in vivo assays such as in vivo high-throughput screening (HTS) assays.

The term “modified versions of these agonists” in accordance with the present invention refers to versions of the agonists that are, e.g., modified if possible to achieve i) modified spectrum of activity, organ specificity, and/or ii) improved potency, and/or iii) decreased toxicity (improved therapeutic index), and/or iv) decreased side effects, and/or v) modified onset of therapeutic action, duration of effect, and/or vi) modified pharmacokinetic parameters (resorption, distribution, metabolism and excretion), and/or vii) modified physico-chemical parameters (solubility, hygroscopicity, color, taste, odor, stability, state), and/or viii) improved general specificity, organ/tissue specificity, and/or ix) optimised application form and route by (a) esterification of carboxyl groups, or (b) esterification of hydroxyl groups with carboxylic acids, or (c) esterification of hydroxyl groups to, e.g. phosphates, pyrophosphates or sulfates or hemi-succinates, or (d) formation of pharmaceutically acceptable salts, or (e) formation of pharmaceutically acceptable complexes, or (f) synthesis of pharmacologically active polymers, or (g) introduction of hydrophilic moieties, or (h) introduction/exchange of substituents on aromates or side chains, change of substituent pattern, or (i) modification by introduction of isosteric or bioisosteric moieties, or (j) synthesis of homologous compounds, or (k) introduction of branched side chains, or (k) conversion of alkyl substituents to cyclic analogues, or (l) derivatisation of hydroxyl groups to ketales, acetales, or (m) N-acetylation to amides, phenylcarbamates, or (n) synthesis of Mannich bases, imines, or (o) transformation of ketones or aldehydes to Schiff's bases, oximes, acetales, ketales, enolesters, oxazolidines, thiazolidines; or combinations thereof.

The term “antibody” as used in accordance with the present invention comprises, for example, polyclonal or monoclonal antibodies. Furthermore, also derivatives or fragments thereof, which still retain the binding specificity, are comprised in the term “antibody”.

Antibody fragments or derivatives comprise, inter alia, Fab or Fab′ fragments as well as Fd, F(ab′)2, Fv or scFv fragments; see, for example Harlow and Lane “Antibodies, A Laboratory Manual”, Cold Spring Harbor Laboratory Press, 1988 and Harlow and Lane “Using Antibodies: A Laboratory Manual” Cold Spring Harbor Laboratory Press, 1999. The term “antibody” also includes embodiments such as chimeric (human constant domain, non-human variable domain), single chain and humanized (human antibody with the exception of non-human CDRs) antibodies.

Various techniques for the production of antibodies are well known in the art and described, e.g. in Harlow and Lane (1988) and (1999), loc. cit. Thus, the antibodies can be produced by peptidomimetics. Further, techniques described for the production of single chain antibodies (see, inter alia, U.S. Pat. No. 4,946,778) can be adapted to produce single chain antibodies specific for the target of this invention. Also, transgenic animals or plants (see, e.g., U.S. Pat. No. 6,080,560) may be used to express (humanized) antibodies specific for the target of this invention. Most preferably, the antibody is a monoclonal antibody, such as a human or humanized antibody. For the preparation of monoclonal antibodies, any technique which provides antibodies produced by continuous cell line cultures can be used. Examples for such techniques are described, e.g. in Harlow and Lane (1988) and (1999), loc. cit. and include the hybridoma technique (originally described by Köhler and Milstein Nature 256 (1975), 495-497), the trioma technique, the human B-cell hybridoma technique (Kozbor, Immunology Today 4 (1983), 72) and the EBV-hybridoma technique to produce human monoclonal antibodies (Cole et al., Monoclonal Antibodies and Cancer Therapy, Alan R. Liss, Inc. (1985), 77-96). Surface plasmon resonance as employed in the BIAcore system can be used to increase the efficiency of phage antibodies which bind to a target (Schier, Human Antibodies Hybridomas 7 (1996), 97-105; Malmborg, J. Immunol. Methods 183 (1995), 7-13). It is also envisaged in the context of this invention that the term “antibody” comprises antibody constructs which may be expressed in cells, e.g. antibody constructs which may be transfected and/or transduced via, inter alia, viruses or plasmid vectors.

Preferably, and as outlined herein above for agonists of the secretin receptor in general, the antibody binds to the secretin receptor or interacts with cellular molecules upstream of the secretin receptor to exert its agonistic effect.

As used herein the term “antibody mimetic” refers to a compound which, like an antibody, can specifically bind antigens, but which is not structurally related to antibodies. Antibody mimetics are usually artificial peptides or proteins with a molar mass of about 3 to 20 kDa. An “antibody mimetic” may be an adhiron (which is build on a cystatin scaffold), an adnectin (based on the tenth type III domain of human fibronectin), an affibody (which is based on the Z-domain of staphylococcal protein A), an affilin (structurally derived from gamma-B crystalline or ubiquitin), an anticalin (derived from lipocalins), an avimer (based e.g. on multimerised Low Density Lipoprotein Receptor (LDLR)-A), a DARPin (derived from ankyrin repeat proteins), a Fynomer® (which is derived from the human Fyn SH3 domain), a Kunitz domain peptide (derived from the Kunitz domains of various protease inhibitors) or a nanofitin (derived from the DNA binding protein Sac7d of Sulfolobus acidocaldarius) (see e.g. Grabulovski et al. (2007) JBC, 282, p. 3196-3204, WO 2008/022759, Bertschinger et al (2007) Protein Eng Des Sel 20(2):57-68, Gebauer and Skerra (2009) Curr Opinion in Chemical Biology 13:245-255, or Schlatter et al. (2012), MAbs 4:4, 1-12). These polypeptides are well known in the art and will be described in further detail herein below. As used herein the term “adhiron” refers to a recombinant non-antibody scaffold protein exhibiting a compact cystatin-like fold and extended flexible regions mediating the contact with the target molecule. Adhirons are commercially known as “affimers”. In order to create an adhiron having a target-specific binding surface, designed or random peptides can be inserted in two loops within the adhiron as well as at its amino terminus. An adhiron with the desired target specificity and affinity can be identified by generating and screening a phage display library of randomised potential target interaction sequences (Tiede et al. (2014) Protein Eng Des Sel 27(5):145-55) The term “adhiron” as used herein also encompasses multimeric forms of adhiron proteins.

An “adnectin” (also referred to as “monobody”) as used herein, is based on the 10th extracellular domain of human fibronectin III (10Fn3), which adopts an Ig-like β-sandwich fold of 94 residues with 2 to 3 exposed loops, but lacks the central disulphide bridge (Gebauer and Skerra (2009) Curr Opinion in Chemical Biology 13:245-255). Adnectins with the desired target specificity can be genetically engineered by introducing modifications in specific loops of the protein.

The term “affibody” as used herein refers to a family of antibody mimetics which is derived from the Z-domain of staphylococcal protein A. Structurally, affibody molecules are based on a three-helix bundle domain which can also be incorporated into fusion proteins. In itself, an affibody has a molecular mass of around 6 kDa and is stable at high temperatures and under acidic or alkaline conditions. Target specificity is obtained by randomisation of 13 amino acids located in two alpha-helices involved in the binding activity of the parent protein domain (Feldwisch J, Tolmachev V.; (2012) Methods Mol Biol. 899:103-26).

The term “affilin” as used herein refers to antibody mimetics that are developed by using either gamma-B crystalline or ubiquitin as a scaffold and modifying amino-acids on the surface of these proteins by random mutagenesis. Selection of affilins with the desired target specificity is effected, for example, by phage display or ribosome display techniques. Depending on the scaffold, affilins have a molecular weight of approximately 10 or 20 kDa. As used herein the term affilin also refers to di- or multimerised forms of affilins (Weidle U H, et al., (2013), Cancer Genomics Proteomics; 10(4):155-68).

The term “anticalin” as used herein refers to an engineered protein derived from a lipocalin (Beste G, Schmidt F S, Stibora T, Skerra A. (1999) Proc Natl Acad Sci U S A. 96(5):1898-903; Gebauer and Skerra (2009) Curr Opinion in Chemical Biology 13:245-255). Anticalins possess an eight-stranded β-barrel which forms a highly conserved core unit among the lipocalins and naturally forms binding sites for ligands by means of four structurally variable loops at the open end. Anticalins, although not homologous to the IgG superfamily, show features that so far have been considered typical for the binding sites of antibodies: (i) high structural plasticity as a consequence of sequence variation and (ii) elevated conformational flexibility, allowing induced fit to targets with differing shape.

The term “avimer” as used herein refers to a class of antibody mimetics which consist of two or more peptide sequences of 30 to 35 amino acids each which are derived from A-domains of various membrane receptors and which are connected by linker peptides. Binding of target molecules occurs via the A-domain and domains with the desired binding specificity can, for example, be selected by phage display techniques. Binding specificity of the different A-domains contained in an avimer may, but does not have to, be identical (Weidle U H, et al., (2013), Cancer Genomics Proteomics; 10(4):155-68).

As used herein, the term “DARPin” refers to a designed ankyrin repeat domain (166 residues), which provides a rigid interface arising from typically three repeated β-turns. DARPins usually carry three repeats corresponding to an artificial consensus sequence, whereby six positions per repeat are randomised. Consequently, DARPins lack structural flexibility (Gebauer and Skerra, 2009).

As used herein the term “Fynomer®” refers to a non-immunoglobulin-derived binding polypeptide derived from the human Fyn SH3 domain. Fyn SH3-derived polypeptides are well-known in the art and have been described e.g. in Grabulovski et al. (2007) JBC, 282, p. 3196-3204, WO 2008/022759, Bertschinger et al (2007) Protein Eng Des Sel 20(2):57-68, Gebauer and Skerra (2009) Curr Opinion in Chemical Biology 13:245-255, or Schlatter et al. (2012), MAbs 4:4, 1-12).

A “Kunitz domain peptide” is derived from the Kunitz domain of a Kunitz-type protease inhibitor such as bovine pancreatic trypsin inhibitor (BPTI), amyloid precursor protein (APP) or tissue factor pathway inhibitor (TFPI). Kunitz domains have a molecular weight of approximately 6 kDA and domains with the required target specificity can be selected by display techniques such as phage display (Weidle et al., (2013), Cancer Genomics Proteomics;10(4):155-68).

A “nanofitin” (also known as affitin) is an antibody mimetic protein that is derived from the DNA binding protein Sac7d of Sulfolobus acidocaldarius. Nanofitins usually have a molecular weight of around 7 kDa and are designed to specifically bind a target molecule by randomising the amino acids on the binding surface (Mouratou B, Behar G, Paillard-Laurance L, Colinet S, Pecorari F., (2012) Methods Mol Biol.; 805:315-31).

Aptamers are nucleic acid molecules or peptide molecules that bind a specific target molecule. Aptamers are usually created by selecting them from a large random sequence pool, but natural aptamers also exist in riboswitches. Aptamers can be used for both basic research and clinical purposes as macromolecular drugs. Aptamers can be combined with ribozymes to self-cleave in the presence of their target molecule. These compound molecules have additional research, industrial and clinical applications (Osborne et. al. (1997), Current Opinion in Chemical Biology, 1:5-9; Stull & Szoka (1995), Pharmaceutical Research, 12, 4:465-483).

More specifically, aptamers can be classified as nucleic acid aptamers, such as DNA or RNA aptamers, or peptide aptamers. Whereas the former normally consist of (usually short) strands of oligonucleotides, the latter preferably consist of a short variable peptide domain, attached at both ends to a protein scaffold.

Nucleic acid aptamers are nucleic acid species that, as a rule, have been engineered through repeated rounds of in vitro selection or equivalently, SELEX (systematic evolution of ligands by exponential enrichment) to bind to various molecular targets such as small molecules, proteins, nucleic acids, and even cells, tissues and organisms.

Peptide aptamers usually are peptides or proteins that are designed to interfere with other protein interactions inside cells. They consist of a variable peptide loop attached at both ends to a protein scaffold. This double structural constraint greatly increases the binding affinity of the peptide aptamer to levels comparable to an antibody's (nanomolar range). The variable peptide loop typically comprises 10 to 20 amino acids, and the scaffold may be any protein having good solubility properties. Currently, the bacterial protein Thioredoxin-A is the most commonly used scaffold protein, the variable peptide loop being inserted within the redox-active site, which is a -Cys-Gly-Pro-Cys- loop in the wild protein, the two cysteins lateral chains being able to form a disulfide bridge. Peptide aptamer selection can be made using different systems, but the most widely used is currently the yeast two-hybrid system. Aptamers offer the utility for biotechnological and therapeutic applications as they offer molecular recognition properties that rival those of the commonly used biomolecules, in particular antibodies. In addition to their discriminate recognition, aptamers offer advantages over antibodies as they can be engineered completely in a test tube, are readily produced by chemical synthesis, possess desirable storage properties, and elicit little or no immunogenicity in therapeutic applications. Non-modified aptamers are usually cleared rapidly from the bloodstream, with a half-life of minutes to hours, mainly due to nuclease degradation and clearance from the body by the kidneys, a result of the aptamer's inherently low molecular weight. Unmodified aptamer applications currently focus on treating transient conditions such as blood clotting, or treating organs such as the eye where local delivery is possible. This rapid clearance can be an advantage in applications such as in vivo diagnostic imaging. Several modifications, such as 2′-fluorine-substituted pyrimidines, polyethylene glycol (PEG) linkage, fusion to albumin or other half life extending proteins etc. are available to scientists such that the half-life of aptamers can be increased for several days or even weeks.

Preferably, and as outlined herein above for agonists of the secretin receptor in general, the aptamer binds to the secretin receptor or interacts with cellular molecules upstream of the secretin receptor to exert its agonistic effect.

The term “peptide” as used herein describes a group of molecules consisting of up to 30 amino acids, whereas the term “polypeptide” as used herein describes a group of molecules consisting of more than 30 amino acids. The group of peptides and polypeptides are referred to together with the term “(poly)peptide”. Also encompassed by the term “(poly)peptide” are proteins as well as fragments of proteins of more than 30 amino acids. The term “fragment of protein” in accordance with the present invention refers to a portion of a protein comprising at least the amino acid residues necessary to maintain the biological activity of the protein. Preferably, the amino acid chains are linear. (Poly)peptides may further form multimers consisting of at least two identical or different molecules. The corresponding higher order structures of such multimers are correspondingly termed homo- or heterodimers, homo- or heterotrimers etc. Furthermore, peptidomimetics of such (poly)peptides where amino acid(s) and/or peptide bond(s) have been replaced by functional analogues are also encompassed by the invention. Such functional analogues include all known amino acids other than the 20 gene-encoded amino acids, such as selenocysteine. The term “(poly)peptide” also refers to naturally modified (poly)peptides where the modification is effected e.g. by glycosylation, acetylation, phosphorylation and similar modifications which are well known in the art.

It is also well known that (poly)peptides are not always entirely linear. For instance, (poly)peptides may be branched as a result of ubiquitination, and they may be circular, with or without branching, generally as a result of post-translation events, including natural processing event and events brought about by human manipulation which do not occur naturally. Circular, branched and branched circular (poly)peptides may be synthesized by non-translational natural processes and by synthetic methods. The modifications can be a function of how the (poly)peptide is made. For recombinant (poly)peptides, for example, the modifications will be determined by the host cells posttranslational modification capacity and the modification signals in the amino acid sequence. Accordingly, when glycosylation is desired, a (poly)peptide should be expressed in a glycosylating host, generally an eukaryotic cell, for example Cos7, HELA or others. The same type of modification may be present in the same or varying degree at several sites in a given (poly)peptide. Also, a given (poly)peptide may contain more than one type of modification.

In accordance with the present invention, the term “small interfering RNA (siRNA)”, also known as short interfering RNA or silencing RNA, refers to a class of 18 to 30, preferably 19 to 25, most preferred 21 to 23 or even more preferably 21 nucleotide-long double-stranded RNA molecules that play a variety of roles in biology. Most notably, siRNA is involved in the RNA interference (RNAi) pathway where the siRNA interferes with the expression of a specific gene. In addition to their role in the RNAi pathway, siRNAs also act in RNAi-related pathways, e.g. as an antiviral mechanism or in shaping the chromatin structure of a genome.

siRNAs naturally found in nature have a well defined structure: a short double-strand of RNA (dsRNA) with 2-nt 3′ overhangs on either end. Each strand has a 5′ phosphate group and a 3′ hydroxyl (—OH) group. This structure is the result of processing by dicer, an enzyme that converts either long dsRNAs or small hairpin RNAs into siRNAs. siRNAs can also be exogenously (artificially) introduced into cells to bring about the specific knockdown of a gene of interest. Essentially any gene of which the sequence is known can thus be targeted based on sequence complementarity with an appropriately tailored siRNA. The double-stranded RNA molecule or a metabolic processing product thereof is capable of mediating target-specific nucleic acid modifications, particularly RNA interference and/or DNA methylation. Exogenously introduced siRNAs may be devoid of overhangs at their 3′ and 5′ ends, however, it is preferred that at least one RNA strand has a 5′- and/or 3′-overhang. Preferably, one end of the double-strand has a 3′-overhang from 1-5 nucleotides, more preferably from 1-3 nucleotides and most preferably 2 nucleotides. The other end may be blunt-ended or has up to 6 nucleotides 3′-overhang. In general, any RNA molecule suitable to act as siRNA is envisioned in the present invention. The most efficient silencing was so far obtained with siRNA duplexes composed of 21-nt sense and 21-nt antisense strands, paired in a manner to have a 2-nt 3′-overhang. The sequence of the 2-nt 3′ overhang makes a small contribution to the specificity of target recognition restricted to the unpaired nucleotide adjacent to the first base pair (Elbashir et al. 2001). 2′-deoxynucleotides in the 3′ overhangs are as efficient as ribonucleotides, but are often cheaper to synthesize and probably more nuclease resistant. Delivery of siRNA may be accomplished using any of the methods known in the art, for example by combining the siRNA with saline and administering the combination intravenously or intranasally or by formulating siRNA in glucose (such as for example 5% glucose) or cationic lipids and polymers can be used for siRNA delivery in vivo through systemic routes either intravenously (IV) or intraperitoneally (IP) (Fougerolles et al. (2008), Current Opinion in Pharmacology, 8:280-285; Lu et al. (2008), Methods in Molecular Biology, vol. 437: Drug Delivery Systems—Chapter 3: Delivering Small Interfering RNA for Novel Therapeutics). The activity and specificity of siRNAs can be altered by various modifications such as, e.g., by inclusion of a blocking group at the 3′ and 5′ ends, wherein the term “blocking group refers to substituents of that can be attached to oligonucleotides or nucleomonomers, either as protecting groups or coupling groups for synthesis (cf. WO 98/13526, EP 2221377 B1), by inclusion of agents that enhance the affinity to the target sequence such as intercalating agents (e.g., acridine, chlorambucil, phenazinium, benzophenanthirdine), attaching a conjugating or complexing agent or encapsulating it to facilitate cellular uptake, or attaching targeting moieties for targeted delivery. As mentioned above, the siRNAs of SEQ ID NOs 8, 9 and 10 were used in example 10 (see also FIG. 10E) in order to silence the mRNA encoding the secretin receptor. For this reason SEQ ID NOs 8, 9 and 10 are particularly preferred siRNAs to be used in the context of the present invention.

A short hairpin RNA (shRNA) is a sequence of RNA that makes a tight hairpin turn that can be used to silence gene expression via RNA interference. shRNA uses a vector introduced into cells and utilizes the U6 promoter to ensure that the shRNA is always expressed. This vector is usually passed on to daughter cells, allowing the gene silencing to be inherited. The shRNA hairpin structure is cleaved by the cellular machinery into siRNA, which is then bound to the RNA-induced silencing complex (RISC). This complex binds to and cleaves mRNAs which match the siRNA that is bound to it. si/shRNAs to be used in the present invention are preferably chemically synthesized using appropriately protected ribonucleoside phosphoramidites and a conventional DNA/RNA synthesizer. Suppliers of RNA synthesis reagents are Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA), and Cruachem (Glasgow, UK). Most conveniently, siRNAs or shRNAs are obtained from commercial RNA oligo synthesis suppliers, which sell RNA-synthesis products of different quality and costs. In general, the RNAs applicable in the present invention are conventionally synthesized and are readily provided in a quality suitable for RNAi.

Further molecules effecting RNAi include, for example, microRNAs (miRNA). Said RNA species are single-stranded RNA molecules which, as endogenous RNA molecules, regulate gene expression. Binding to a complementary mRNA transcript triggers the degradation of said mRNA transcript through a process similar to RNA interference.

Accordingly, siRNA, an shRNA or a miRNA may be useful as agonists that have an indirect agonistic effect on the secretin receptor such as, e.g., by facilitating the degradation of a repressor of expression and/or release of the natural ligand secretin or a repressor of the expression of or the activity of the secretin receptor. Accordingly, and as detailed herein above, a Agtr1-specific siRNA, an shRNA or a miRNA resulting in a decrease of Agtr1 activity is a preferred secretin receptor agonist.

A ribozyme (from ribonucleic acid enzyme, also called RNA enzyme or catalytic RNA) is an RNA molecule that catalyzes a chemical reaction. Many natural ribozymes catalyze either their own cleavage or the cleavage of other RNAs, but they have also been found to catalyze the aminotransferase activity of the ribosome. Non-limiting examples of well-characterized small self-cleaving RNAs are the hammerhead, hairpin, hepatitis delta virus, and in vitro-selected lead-dependent ribozymes, whereas the group I intron is an example for larger ribozymes. The principle of catalytic self-cleavage has become well established in the last 10 years. The hammerhead ribozymes are characterized best among the RNA molecules with ribozyme activity. Since it was shown that hammerhead structures can be integrated into heterologous RNA sequences and that ribozyme activity can thereby be transferred to these molecules, it appears that catalytic antisense sequences for almost any target sequence can be created, provided the target sequence contains a potential matching cleavage site. The basic principle of constructing hammerhead ribozymes is as follows: An interesting region of the RNA, which contains the GUC (or CUC) triplet, is selected. Two oligonucleotide strands, each usually with 6 to 8 nucleotides, are taken and the catalytic hammerhead sequence is inserted between them. Molecules of this type were synthesized for numerous target sequences. They showed catalytic activity in vitro and in some cases also in vivo. The best results are usually obtained with short ribozymes and target sequences.

A recent development, also useful in accordance with the present invention, is the combination of an aptamer recognizing a small compound with a hammerhead ribozyme. The conformational change induced in the aptamer upon binding the target molecule is supposed to regulate the catalytic function of the ribozyme.

The term “antisense nucleic acid molecule” is known in the art and refers to a nucleic acid which is complementary to a target nucleic acid. An antisense molecule in accordance with the invention is capable of interacting with the target nucleic acid, more specifically it is capable of hybridizing with the target nucleic acid. Due to the formation of the hybrid, transcription of the target gene(s) and/or translation of the target mRNA is reduced or blocked. Standard methods relating to antisense technology have been described (see, e.g., Melani et al., Cancer Res. (1991) 51:2897-2901). Accordingly, an antisense molecule can exert an agonistic effect on the secretin receptor as outlined herein above for the siRNA, shRNA and miRNA agonists.

The hormone “secretin” as the natural ligand for the secretin receptor is well-known in the art, has been described herein above in detail and is used in accordance herewith.

A secretin “fragment” in accordance with the invention is meant to relate to a partial secretin peptide sequence, i.e. not the entire secretin peptide sequence, wherein said partial secretin peptide sequence maintains/has the function of full length secretin sequence which is activating the secretin receptor resulting in an increase of non-shivering thermogenesis in brown adipocytes and/or in an increase of the expression of UCP1 in brown adipocytes. As such, it can also be referred to as a “functional fragment” of secretin. It is well known in the art that functional polypeptides may be cleaved to yield fragments with unaltered or substantially unaltered function. Such cleavage may include the removal of a given number of N- and/or C-terminal amino acids. Additionally or alternatively, a number of internal (non-terminal) amino acids may be removed, provided the obtained polypeptide has the above described activity. Said number of amino acids to be removed from the termini and/or internal regions may be one, two, three, four, five, six, seven, eight, nine, ten or more. In particular, removals of amino acids which preserve sequence and boundaries of any conserved functional domain(s) or subsequences in the secretin sequence are particularly envisaged. Means and methods for determining such domains are well known in the art and include experimental and bioinformatic means. Experimental means include the systematic generation of deletion mutants and their assessment in assays for determining non-shivering thermogenesis and/or expression of UCP1 known in the art and as described in the Examples enclosed herewith (see above and example 1). Bioinformatic means include database searches. Suitable databases included protein sequence databases. In this case a multiple sequence alignment of significant hits is indicative of domain boundaries, wherein the domain(s) is/are comprised of the/those subsequences exhibiting an elevated level of sequence conservation as compared to the remainder of the sequence. Further suitable databases include databases of statistical models of conserved protein domains such as Pfam maintained by the Sanger Institute, UK (www.sanger.ac.uk/Software/Pfam). A corresponding fragment may also be part of a larger (poly)-peptide sequence, wherein the additional (poly)-peptide sequence/s is/are not originating from secretin.

A secretin “analogue” (or also termed “derivative” herein) in accordance with the invention relates to a secretin sequence in which one or more amino acids may be deleted, modified, inserted and/or substituted. It is understood that the analogue/derivative is functional. The derivative is functional, if it is capable of activating the secretin receptor resulting in an increase of non-shivering thermogenesis in brown adipocytes and/or in an increase of the expression of UCP1 in brown adipocytes. Furthermore, in the context of a “functional derivative”, an insertion refers to the insertion of one or more amino acids into the original secretin sequence. It is preferred with increasing preference that a functional derivative does not comprise more than 10, 9, 8, 7, 6, 5, 4, 3, 2, or nor more than 1 amino acid change(s) (i.e. deleted, modified, inserted and/or substituted amino acids). In another embodiment, it is preferred with increasing preference that not more than 20%, 15%, 10%, 9%, 8%, 7%, 6%, 5%, 4%, 3%, 2%, or not more than 1% of all amino acids of the secretin sequence are changed (i.e. are deleted, modified, inserted and/or substituted amino acids). A substitution in a derivative may be a conservative or a non-conservative substitution, but preferably is a conservative substitution. In some embodiments, a substitution also includes the exchange of a naturally occurring amino acid with a not naturally occurring amino acid. A conservative substitution comprises the substitution of an amino acid with another amino acid having a chemical property similar to the amino acid that is substituted. Preferably, the conservative substitution is a substitution selected from the group consisting of: (i) a substitution of a basic amino acid with another, different basic amino acid; (ii) a substitution of an acidic amino acid with another, different acidic amino acid; (iii) a substitution of an aromatic amino acid with another, different aromatic amino acid; (iv) a substitution of a non-polar, aliphatic amino acid with another, different non-polar, aliphatic amino acid; and (v) a substitution of a polar, uncharged amino acid with another, different polar, uncharged amino acid. A basic amino acid is selected from the group consisting of arginine, histidine, and lysine. An acidic amino acid is selected from aspartate or glutamate. An aromatic amino acid is selected from the group consisting of phenylalanine, tyrosine and tryptophane. A non-polar, aliphatic amino acid is selected from the group consisting of glycine, alanine, valine, leucine, methionine and isoleucine. A polar, uncharged amino acid is selected from the group consisting of serine, threonine, cysteine, proline, asparagine and glutamine. In contrast to a conservative amino acid substitution, a non-conservative amino acid substitution is the exchange of one amino acid with any amino acid that does not fall under the above-outlined conservative substitutions (i) through (v). If a functional derivative comprises a deletion, then in the derivative one or several amino acids that are present in the reference polypeptide or amino acid sequence have been removed. The deletion may, however, not be so extensive that the derivative comprises less than 20, preferably less than 18, more preferably less than 16 and most preferably less than 12 amino acids in total. As mentioned above, amino acids of secretin may also be modified, e.g. chemically modified. For example, the side chain or a free amino or carboxy-terminus of an amino acid of the polypeptide may be modified by e.g. glycosylation, amidation, phosphorylation, ubiquitination, e.t.c. The chemical modification can also take place in vivo, e.g. in a host-cell, as is well known in the art. For examples, a suitable chemical modification motif, e.g. glycosylation sequence motif present in the amino acid sequence of the polypeptide will cause the polypeptide to be glycosylated. Examples of analogues are [Tyr10, pNO2-Phe22]secretin-27 (see Ganguli et al., 1998, The Journal of pharmacology and experimental therapeutics 286: 593-598) and COOH-terminally extended forms of secretin (see Solomon et al., 1999, The American journal of physiology 276: G808-816).

A “stimulant of secretin release” relates in accordance with the invention to an agonist of the secretin receptor that triggers or increases the release of secretin. It is understood that a stimulant of secretin release as agonist of the secretin receptor in accordance with the invention will only be effective in a scenario where the released secretin can bind to a secretin receptor on the brown adipocytes. A corresponding scenario will be any embodiment of a method of the invention performed in vivo method and, in particular the embodiment relating to the agonist of the secretin receptor for use in the prevention and/or treatment of a disease or disorder of energy homeostasis. Thus, an embodiment concerning an ex vivo or in vitro method of the invention does not encompass the use of a corresponding agonist of the secretin receptor. An example of a corresponding stimulant is licorice extract as described in Shiratori et al. (Pancreas 1: 483-487 (1986)). Briefly, the licorice extract, known as FM100, was obtained by precipitating a methanolic extract with sodium hydroxide and hydrochloric acid. Further examples are candidates for secretin releasing peptides (SRP) such as pituitary adenylate cyclase activating peptide (PACAP) and phospholipase A2 (PLA2) as described in Chey and Chang. 2003, Journal of physiology and pharmacology: an official journal of the Polish Physiological Society 54 Suppl 4: 105-112; Chang et al., 1999, Pancreas 19: 401-405; Chey and Chang, 2014, Pancreas 43(2):162-182; and Li et al., 1990, The Journal of clinical investigation 86: 1474-1479.

1-Phenylpentanol is a further stimulant (see Chey and Chang, 2003, Journal of gastroenterology 38: 1025-1035). MCI-727 (CAS Registry Number 122378-96-5), geranyl-geranyl acetone (also known as Teprenone; described in Ebas et al., 1990, Pancreas 5: 555-558) and Plaunotol (CAS Registry Number 64218-02-6) described in Shiratori et al., 1989, Pancreas 4: 323-328; Shiratori et al., 1989, Digestive diseases and sciences 35: 1140-1145 and originally known as an anti-ulcer agents are further stimulants. Camostat (CAS Registry Number 59721-29-8), a synthetic trypsin inhibitor as also shown to be a stimulant (see Watanabe et al., 1992, Gastroenterology 102: 621-628). Also, secretin release is positively regulated by PACAP (Pituitary adenylate cyclase-activating polypeptide), gastrin-releasing peptide (GRP) or serotonin (see Lee et al., 1998, Gastroenterology 114: 1054-1060; Li et al., 2001, American journal of physiology Gastrointestinal and liver physiology 280: G595-602; Park et al., 2000, American journal of physiology Gastrointestinal and liver physiology 278: G557-562) which are, thus, stimulants of secretin release and, therefore, agonists of the secretin receptor in accordance with the invention.

The agonists described in this embodiment are understood to also include modified versions of said agonists.

In accordance with the invention, it is understood that the test compounds used in accordance with the method for identifying agonists of the secretin receptor can belong to any of the above compound classes. Thus, the above definitions and explanations apply mutatis mutandis also to the method of the invention for identifying agonists of the secretin receptor.

In a preferred embodiment of the different embodiments of the invention, the antagonist of the secretin receptor is selected from the group consisting of an antibody or a fragment or derivative thereof, an antibody mimetic, an aptamer, an siRNA, an shRNA, a miRNA, a ribozyme, an antisense nucleic acid molecule, or a small molecule.

The definitions, explanations and preferred embodiments described above in relations to agonists of the secretin receptor apply mutatis mutandis also to this embodiment.

Examples of antagonists of the secretin receptor are Secretin[5-27] analogue with lactam connecting residues 16 and 20 [c[E(16), K(20)][Y(10)]sec(5-27)] (see Dong et al., 2011. Biochemistry 50: 8181-8192), Sec[5-27] analogues as described, e.g., in Te et al., 2012, J Comput Aided Mol Des 26: 835-845; Miegueu et al., 2013, International journal of obesity 37: 366-374), and cimetidine (CAS Registry Number 51481-61-9) described in Kim et al., 1979, The American journal of physiology 236: E539-544) or other suppressors of gastric acid secretion resulting in suppression of secretin release after meals. Further exemplary antagonists are described in the example section and include the specifically mentioned siRNAs and the protein kinase inhibitor H89. While described as exemplary antagonists, they are also preferred antagonist for all embodiments of the invention.

In accordance with the invention, it is understood that the test compounds used in accordance with the method for identifying antagonists of the secretin receptor can belong to any of the above compound classes. Thus, the above definitions and explanations apply mutatis mutandis also to the method of the invention for identifying antagonists of the secretin receptor.

As regards the embodiments characterized in this specification, in particular in the claims, it is intended that each embodiment mentioned in a dependent claim is combined with each embodiment of each claim (independent or dependent) said dependent claim depends from. For example, in case of an independent claim 1 reciting 3 alternatives A, B and C, a dependent claim 2 reciting 3 alternatives D, E and F and a claim 3 depending from claims 1 and 2 and reciting 3 alternatives G, H and I, it is to be understood that the specification unambiguously discloses embodiments corresponding to combinations A, D, G; A, D, H; A, D, I; A, E, G; A, E, H; A, E, I; A, F, G; A, F, H; A, F, I; B, D, G; B, D, H; B, D, I; B, E, G; B, E, H; B, E, I; B, F, G; B, F, H; B, F, I; C, D, G; C, D, H; C, D, I; C, E, G; C, E, H; C, E, I; C, F, G; C, F, H; C, F, I, unless specifically mentioned otherwise.

Similarly, and also in those cases where independent and/or dependent claims do not recite alternatives, it is understood that if dependent claims refer back to a plurality of preceding claims, any combination of subject-matter covered thereby is considered to be explicitly disclosed. For example, in case of an independent claim 1, a dependent claim 2 referring back to claim 1, and a dependent claim 3 referring back to both claims 2 and 1, it follows that the combination of the subject-matter of claims 3 and 1 is clearly and unambiguously disclosed as is the combination of the subject-matter of claims 3, 2 and 1. In case a further dependent claim 4 is present which refers to any one of claims 1 to 3, it follows that the combination of the subject-matter of claims 4 and 1, of claims 4, 2 and 1, of claims 4, 3 and 1, as well as of claims 4, 3, 2 and 1 is clearly and unambiguously disclosed.

The above considerations apply mutatis mutandis to all attached claims.

The figures show:

FIG. 1: Secretin receptor expression in mouse adipose tissues (A) and in differentiated primary adipocytes (B).

A: Relative expression of secretin receptor (Sctr) in mouse brown adipose tissue (BAT), subcutaneous inguinal (iWAT) and intra abdominal epidydimal (eWAT) white adipose tissue depots normalized to transcription factor TFIIB for N=5. Statistics were conducted using one-way ANOVA and since normality test (Shapiro-Wilk) failed (P<0.050) a Kruskal-Wallis one-way analysis of variance on ranks was performed (p=0.021). The * indicates p<0.05.

B: Expression of secretin receptor in primary adipocytes was also normalized by TFIIB-expression. Data are presented as mean of technical triplicates of N=1.

FIG. 2: Secretin induced oxygen consumption rates (OCR) in brown adipocytes.

Cell respiration measurements were conducted on the seventh day of differentiation.

A: One representative measurement is shown in technical replicates of 6-7; Dots represent means±SD. Microplate wells with differentiated brown adipocytes were injected subsequently with oligomycin (Oligo), secretin/isoproterenol (Sct/Iso), FCCP and antimycin A (Anti A).

B: Percentage fold increase above basal respiration (p=0.712).

C: Percentage attainment of maximal uncoupled FCCP respiration (p=0.946). In B and C values of three individual measurements with technical replicates of 6-8 per group were used and are represented as dots. Significance was verified by t-test.

FIG. 3: Secretin induced oxygen consumption in brown adipocytes of UCP1 knockout mice.

Measurement of oxygen consumption rates (OCR) was conducted at the seventh day of differentiation. Microplate wells with differentiated brown adipocytes were injected with oligomycin (Oligo), secretin/isoproterenol (Sct/Iso), FCCP and antimycin A (Anti A). Dots represent means±SD of 6-16 technical replicates.

A: treatment with secretin (Sct)

B: treatment with isoproterenol (Iso).

FIG. 4: Secretin induced UCP1 expression in primary adipocytes.

Differentiated primary adipocytes from inguinal white adipose tissue (A) or brown adipose tissue (B) were stimulated with 0.1 μM secretin or 0.5 μM isoproterenol for 6 hours. Expression of UCP1 mRNA was measured by qRT-PCR and normalized to TFIIB expression. Results were standardized to PBS treated condition. Bars represent means (N=3) and dots denote single measurements, each consisting of technical triplicates. For statistical analysis one-way ANOVA was conducted (WAT: PBS vs Sct p<0.001; PBS vs Iso p<0.001; Sct vs Iso p=0.066; BAT: PBS vs Sct p=0.008; PBS vs Iso p=0.018; Sct vs Iso p=0.291).

FIG. 5: Secretin induced respiration in wildtype 129/S6 mice.

Mice received 0.5 mg/kg secretin solved in PBS or an equal volume of PBS via i.p. injection.

A: Measurement was performed at 27° C. The mean of N=7 mice per group is shown for heat production (HP). The Mean of basal HP measured at 30° C. is depicted as grey background).

B: AUC for heat production for 45 minutes of measurement (p=0.001). For statistical analysis the basal heat production was subtracted for every mouse individually. Statistical analysis was performed using t-test.

FIG. 6: Secretin induced heat production in UCP1 knockout mice.

Mice received 0.5 mg/kg secretin solved in PBS or equal volume of PBS via i.p. injection. Measurement was performed at 27° C. In (A) the mean of N=9 wildtype mice per group and in (B) the mean of N=5-6 knockout mice are shown for heat production (mean of basal HP measured at 30° C. depicted as grey background). In (C) the AUC for heat production for 60 minutes of measurement is depicted. For statistical analysis the basal heat production was subtracted for every mouse individually. Statistics were conducted using two-way ANOVA. Equal variance test failed (P<0.050) and therefore a pairwise multiple comparison was conducted using Tukey test (genotype: p=0.005; treatment: p=0.013; genotype×treatment: p=0.128).

There was a significant difference for the treatment within wildtype mice (p=0.002; indicated by **) but not within knockout mice (p=0.482) and furthermore a significant difference for the genotype within secretin treatment (p=0.005), but not within PBS treatment (p=0.275), which is not illustrated in the graph.

FIG. 7: Effect of secretin on refeeding of fasted mice.

Mice fasted for 18 h were injected with 5 nmol secretin before the start of refeeding. Control mice were injected with PBS. Food intake is shown for 4 hours after injection. Data are presented as mean±SD (N=11-12 per group). Statistical analysis were conducted for the first two hours using linear mixed-effects model with TIBICO Spotfire SPlus software (Treatment: p=0.0181; Time: p<0.0001).

FIG. 8: Effect of secretin on refeeding of fasted wildtype and Ucp1 knockout mice.

Wildtype (WT) and Ucp1 knockout (KO) mice fasted for 18 h were injected with 5 nmol secretin (SCT) before start of refeeding. Control mice of both genotypes were injected with PBS. Cumulative food intake was monitored during 4 hours of refeeding. Data are presented as mean of N=6-7 per group.

A: amount of food intake over time;

B: AUC of food intake.

FIG. 9: Sctr gene expression by transcriptome analysis (RNA-SEQ).

A: Secretin receptor (SctR), a Gs coupled GPCR, is highly expressed in BAT. Gene expression of receptors for gastrointestinal tract-related peptides obtained from RNA sequencing of murine brown adipose tissue. Data are shown in RPKM (reads per kilobase per million mapped reads). Expression of secretin receptor (SctR) is significantly higher compared to gastric inhibitory polypeptide receptor (Gipr), vasoactive intestinal peptide receptor1/2 (Vipr1/2), cholecystokinin A/B receptor (Ccka/br), ghrelin receptor (Ghsr), glucagon-like peptide 1 receptor (Glp1r), neuropeptide Y receptor type 2 (Npy2r) (p<0.0001).

B: Secretin receptor gene expression in interscapular BAT, inguinal WAT and gonadal WAT of mice.

C: Secretin receptor gene expression in primary cultured adipocytes from interscapular BAT, inguinal WAT and gonadal WAT of mice.

FIG. 10: Respiration measurements in primary brown adipocytes of wildtype and UCP1−/− mice.

A: Wildtype data: Isoproterenol (500 nM) and Secretin (10 nM) activate UCP1-dependent thermogenesis in primary brown adipocytes cultured from wildtype mice. Data are from five independent experiments (N=5).

B: Knock-out data Isoproterenol (500 nM) and Secretin (10 nM) fail to activate thermogenesis in primary brown adipocytes cultured from UCP1^{-/-} mice. Data are from five independent experiments (N=5).

C: Secretin activates UCP1-dependent thermogenesis in primary murine brown adipocytes. Comparison of isoproterenol (ISO) and secretin (SCT) stimulated respiration in primary brown adipocytes from UCP1^{+/+} and UCP1^{-/-} mice. UCP1-dependent thermogenesis is expressed as fold increase above basal leak respiration. Microplate respirometry of primary brown adipocytes was conducted following the subsequent protocol. After assessment of basal oxygen consumption oligo (oligomycin) was injected to determine basal leak respiration. Next, either ISO or SCT was added to investigate UCP1 (uncoupling protein 1)-dependant uncoupled respiration. By the addition of FCCP maximal leak respiration was determined. Lastly, non-mitochondrial oxygen consumption was assessed by injecting Anti A (antimycin A).

D: SCT induced thermogenesis in brown adipocytes does not depend on beta-adrenergic receptor signaling. SCT- and ISO-stimulated respiration after 1 h pretreatment with different concentrations propranolol, a non-selective blocker of adrenergic β-receptors.

E: Thermogenic effect of SCT depends on secretin receptor expression. SCT- and ISO stimulated respiration in primary brown adipocytes after siRNA-mediated knockdown of the secretin receptor (SCTR) in comparison to a non-targeting control (NC).

F: Thermogenic effect of SCT depends on Protein Kinase A activity. Fold increase of basal leak respiration after stimulation with ISO, SCT and vehicle (assay medium) w/o proteinkinase A inhibitor H89 (50 μM). Inhibitor was injected together with olgio prior to addition of stimulators.

FIG. 11: Plasma SCTR levels are regulated by fasting and refeeding.

Secretin plasma levels were determined in mice after 18 h fasting, and mice fasted for 17 h and refed for 1 h. Control mice were fed ad libitum.

The examples illustrate the invention:

### EXAMPLE 1

Material and Methods:

Animals

Male mice (Mus musculus, Sv129S6/J, Sv129S1/SvImJ and C56BL/6J) were housed at an ambient temperature of 22° C. and a humidity of 50-60% at the animal facility of TUM in Weihenstephan. The mice were maintained in groups of two to five mice in type-II, long cages (360 m²) with a light-dark cycle of 12 hours each. If not stated otherwise mice had ad libitum access to water and food. The chow diet for breeding and maintenance consisted of 58% carbohydrate, 33% protein and 9% fat, given as % of total energy content (17 kJ/g). UCP1-KO mice with C56BL/6J and 129/SvImJ genetic background have been kindly provided by Leslie Kozak from the Pennington Biomedical Research Center (PBRC), Baton Rouge, L .70808.

Primary Adipocytes

For culturing primary adipocytes male 129S1/SvImJ mice (wildtypes or UCP1-KO mice and wildtype littermates) at the age of 5-6 weeks were used for the dissection of inguinal and epidydimal white adipose tissue and interscapular brown adipose tissue depots. The tissue was carefully minced and treated with collagenase for 45 min at 37° C. The homogenate was filtered through a 250 μm nylon mesh and centrifuged at 500 g to collect the stromal vascular fraction (SVF). The SVF cell pellet was rinsed and seeded into XF96 V3-PS cell culture microplate (Seahorse Bioscience). After reaching confluency, induction medium containing 10% fetal bovine serum (FBS), 0.5 mM isobutylmethylxanthine, 125 nM indomethacin, 1 mM dexamethasone, 850 nM insulin and 1 nM T3 was added. After 2 days of induction, cells were maintained in differentiation media (10% FBS, 850 nM insulin and 1 nM T3). Medium was changed every two days. Respiration was measured on day 7 of differentiation. For determination of mRNA levels cells were stimulated at the 7^{th} day of differentiation for 6 hours with secretin (rat, Tocris, Art Nr 1919) or isoproterenol (Sigma-Aldrich, Art Nr. 16504-100MG) and harvested subsequently.

Respirometry

Oxygen consumption rate (OCR) was measured at 37° C. using a microplate respirometer (XF96 extracellular flux analyzer, Seahorse Bioscience). At day 7, the differentiation medium was replaced with prewarmed, unbuffered assay medium (DMEM basal medium supplemented with 25 mM glucose, 2 mM sodium pyruvate, 31 mM NaCl, 2 mM GlutaMax and 15 mg/l phenol red, pH 7.4) containing 2% of essentially fatty acid free bovine serum albumin (BSA), and incubated at 37° C. in a room air incubator for 1 h. Basal respiration was measured in untreated cells. Coupled respiration was inhibited by oligomycin treatment (5 μM). UCP1 mediated uncoupled respiration was determined after stimulation with 0.5 μM secretin (rat, Tocris, Art Nr 1919) or 0.5 μM isoproterenol (Sigma-Aldrich, Art Nr. I6504-100 MG). Maximum respiratory capacity was assessed after FCCP (Sigma-Aldrich) addition (1 μM). Finally, mitochondrial respiration was blocked by antimycin A (Sigma-Aldrich) (5 μM) treatment and the residual OCR was considered to be due to non-mitochondrial reactions. Oxygen consumption rates were calculated using the Seahorse XF-96 software. Data were exported and reconstructed in GraphPad Prism 4.0 software.

For the presentation of the fold increase above basal and maximal uncoupled OCR values were calculated as follows:

For each condition the lowest OCR after antimycin A addition was subtracted from all other values. Respiration rates following oligomycin addition were defined as basal OCR. For induction of OCR by isoproterenol, secretin or FCCP the highest values measured after addition of the respective molecule was used. The “OCR in % of basal OCR” and “OCR in % of FCCP OCR” were calculated as follows:

$$OCR \% basal OCR = \frac{100 * ( Sct - antiA )}{( basal - antiA )} or \frac{100 * ( Iso - antiA )}{( basal - antiA )}$$

$$OCR \% FCCP OCR = \frac{100 * ( Sct - antiA )}{( FCCP - antiA )} or \frac{100 * ( Iso - antiA )}{( FCCP - antiA )}$$

$$OCR \% basal OCR = \frac{100 * ( Sct - antiA )}{( basal - antiA )} or \frac{100 * ( Iso - antiA )}{( basal - antiA )}$$

$$OCR \% FCCP OCR = \frac{100 * ( Sct - antiA )}{( FCCP - antiA )} or \frac{100 * ( Iso - antiA )}{( FCCP - antiA )}$$

Quantitative PCR

Frozen tissue samples [interscapular brown adipose tissue (BAT), inguinal white adipose tissue (iWAT), and intra-abdominal gonadal white adipose tissue (gWAT)] or primary adipocytes harvested from cell culture were homogenized in TRIsure (Bioline, London/UK) according to the manufacturer's instructions. Precipitated RNA was transferred on spin columns (SV Total RNA Isolation System, Promega, Madison WI/USA) and centrifuged for 15 sec at 8,000 g at room temperature. The residual volume was added to the column and centrifuged for 1 min at 12,000 g at room temperature. Further processing was performed according to the manufacturer's protocol. RNA was eluted in 50 μl nuclease-free water and RNA concentration was determined spectrophotometrically (Infinite 200 PRO NanoQuant, Tecan, Männedorf/Switzerland). Reverse transcription into cDNA was performed with 500 ng RNA in a final volume of 10 μl (Quantitect Reverse Transcription Kit, Quiagen, Hilden/Germany). qRT-PCR was conducted on 384 well plates in a total volume of 12.5 μl comprising 6.25 μl SensiMix SYBR No-ROX (Bioline, London/UK), 250 nM forward and reverse primers, and 1 μl template cDNA (LightCycler 480 System, Roche Diagnostics, Rotkreuz/Switzerland). Transcript levels of target genes were normalized to transcription factor 2b (TFIIB) expression. qRT-PCR primers were produced by Eurofins MWG Operon (Ebersberg/Germany).

Primers

UCP1

| | |
|---|---|
| for 5′-GTACACCAAGGAAGGACCGA-3′ | (SEQ ID NO: 2) |
| rev 5′-TTTATTCGTGGTCTCCCAGC-3′ | (SEQ ID NO: 3) |
| Secretin receptor | |
| for 5′-ATGCACCTGTTTGTGTCCTT-3′ | (SEQ ID NO: 4) |
| rev 5′-TAGTTGGCCATGATGCAGTA-3′ | (SEQ ID NO: 5) |

TFIIB

| | |
|---|---|
| for 5′-TGGAGATTTGTCCACCATGA-3′ | (SEQ ID NO: 6) |
| rev 5′-GAATTGCCAAACTCATCAAAACT-3′ | (SEQ ID NO: 7) |

Food Intake Measurements

For the measurement of food intake, a feeding-drinking-activity (FDA) device was used (TSE Systems, Bad Homburg/Germany). During the measurement mice were single-housed in type-III cages. Mice were habituated to the new cage environment and received every morning at 10 a.m. a single daily intraperitoneal (i.p.) PBS-injection for four days ahead of the experiment start. The body weight of the mice was measured every day. On day four day of habituation the food was removed from the cages at 5 p.m., and the mice were food deprived over night with ad libitum access to water. On the next morning, mice were i.p. injected with either PBS as control or secretin (rat, Tocris, Art Nr 1919). The latter was given in different doses. Subsequently the mice were refed ad libitum while monitoring food intake during the following 72 hours. During this refeeding period mice were only disturbed for daily body weight measurements. The values for food intake were cropped in intervals of 5 minutes by automatic weighing of the feeder. Food intake was calculated from the decrease in feeder weight. The results were exported into a spreadsheet calculation table (Excel, Microsoft) and analyzed with a software package for statistics and graphics (GraphPad Prism 4.0, GraphPad Software, San Diego, USA).

Indirect Calorimetric Measurements

Indirect calorimetric measurements in mice were performed using an open flow respirometry system (Phenomaster, TSE Systems, Bad Homburg/Germany). All animals were kept at room temperature or cold-acclimated at 4° C. for 4 days as indicated in the results section. For the gas exchange measurement (O₂ consumption and CO₂ production) mice were placed individually in metabolic cages (type I, 3 liter volume) without food and water and transferred to a climate cabinet (TPK 600, Feutron, Greiz/Germany). The air was continuously pulled through the cages at a flow rate of 33 l/h. For gas analysis a subsample was dried in a cooling trap and analyzed for gas exchange. Mice in the cabinet were preconditioned to 30° C. and the basal metabolic rate (BMR) in the post-absorptive state was measured between 8:00 a.m.-12:00 p.m. The O₂ consumption and CO₂ production of each mouse was analyzed every 5 min over a period of 1 min. After BMR measurements, the cages were removed from the climate cabinet and the cabinet temperature was lowered to 27° C. within 20 min. Mice were injected intraperitoneally with 0.5 mg/kg secretin (rat, Tocris, Art Nr 1919) or PBS as a control. After injection the O₂ consumption and CO₂ production rate of each mouse was recorded for 45-60 min with high-resolution recordings at 10 sec intervals. BMR [ml O₂/h] was defined as the lowest mean of three consecutive oxygen consumption values, which had a coefficient of variation less than 5%. Respiratory exchange ratios (RER) and heat production (HP) were calculated from the following formula: (RER=CO₂ production/O₂ consumption) and (HP[mW]=(4.44+1.43*respiratory exchange ratio)*oxygen consumption [ml/h]).

### EXAMPLE 2

### Secretin Receptor Expression In Vivo and In Vitro

Wildtype Sv129S6/J mice were killed for the sampling of brown adipose tissue BAT, inguinal white adipose tissue (iWAT) and intra-abdominal gonadal white adipose tissue (gWAT) depots. The secretin receptor expression was measured by quantitative RT-PCR. The mean expression level of the receptor was higher in BAT compared to iWAT and gWAT. This difference was significant for the comparison of BAT and iWAT, but not gWAT (FIG. 1A), which is in line with data from BIO-GPS (http://biogps.org/) showing second highest expression for the secretin receptor in BAT amongst all analyzed mouse tissues (see GeneAtlas MOE430, gcrma, Probeset 1443454_at: neuroblast [43.9]>adipose, brown [16.3]>placenta [12.5]>stomach [10.6]> . . . >adipose, white [5.2]).

The expression of the secretin receptor was also analyzed in primary adipocytes. Isolated cells of the stromal vascular fraction from three different mouse adipose tissue depots (BAT, iWAT and gWAT) were cultured and fully differentiated. The quantification of the secretin receptor by qRT-PCR revealed a pattern in line with the expression analysis in tissues. In differentiated primary adipocytes the expression of the secretin receptor was much higher in primary adipocytes grown from BAT as compared to iWAT or gWAT (FIG. 1B).

### EXAMPLE 3

### Secretin Stimulates Oxygen Consumption Rate in Primary Brown Adipocytes

The main function of brown adipocytes is heat production by uncoupled respiration. Since the secretin receptor is preferentially expressed in brown adipocytes (see example 1) the effects of secretin on their thermogenic function were investigated by measurement of oxygen consumption rates (OCR). Brown adipocytes were subsequently treated with oligomycin (“Oligo” which inhibits coupled respiration), secretin (Sct) or isoproterenol (Iso), FCCP (fully uncoupled respiration) and antimycin A (Anti A; non-mitochondrial OCR) after fully differentiated in a microplate respirometer plate (XF-96 flux analyzer, Seahorse Bioscience). Secretin stimulated OCR was comparable to the stimulation in response to the pan-β-adrenergic receptor agonist isoproterenol, which is an established activator of UCP1-mediated thermogenesis (FIG. 2A). Secretin and isoproterenol both increased OCR about 200% above basal OCR (FIG. 2B). These uncoupled OCRs correspond to about 75% of maximal uncoupled OCR induced by FCCP (FIG. 2C). There was no statistical difference between the secretin and isoproterenol stimulated OCRs when expressed in relation to basal (p=0.712) or maximal respiration (p=0.946). Both compounds were used in the same concentration. The potency of secretin to induce mitochondrial respiration in vitro can be considered equal to the classical pan-β-adrenergic receptor agonist isoproterenol.

### EXAMPLE 4

### Primary Brown Adipocytes from UCP1 Knockout Mice Lack Secretin Induced Oxygen Consumption

Secretin is able to induce respiration in brown fat cells. To investigate the dependency of the induction of mitochondrial respiration (see example 3) on the presence of UCP1 the respiration measurement was repeated with primary brown adipocytes from UCP1 knockout and wildtype control mice (129/S1-background). For both, secretin (FIG. 3A) and isoproterenol (FIG. 3B) an increase in oxygen consumption could be found in cells from wildtype mice (FIG. 3). At the same time the induction of respiration by both, secretin and isoprotrenol was lacking in cells from UCP1 knockout mice. It must be mentioned that the cells from UCP1 knockout mice also exhibited a much smaller fully uncoupled respiration by FCCP than the wildtype controls. The results of this experiment clearly point out that the induction of respiration in brown adipocytes by β-adrenergic or secretin stimulation is dependent upon UCP1. The potential for direct activation of UCP1 is established for isoproterenol. In this experiment it was also shown for the peptide hormone secretin.

### EXAMPLE 5

### Secretin Stimulates UCP1 Expression in White and Brown Adipocytes

Differentiated inguinal white and interscapular brown primary adipocytes were stimulated with secretin or isoproterenol. Expression of mRNA was determined with quantitative PCR. Secretin increased the expression of UCP1 in white adipocytes (FIG. 4A). The effect was highly significant and comparable to the induction of UCP1 expression by isoproterenol (PBS vs. Sct: p<0.001; PBS vs. Iso: p<0.001; Sct vs. Iso: p=0.066). This shows that secretin is equally potent as isoproterenol to induce browning in cultured primary white adipocytes. Likewise, in primary brown adipocytes secretin significantly increased the expression of the UCP1 gene compared to the unstimulated PBS control (FIG. 4B; PBS vs Sct p=0.008; PBS vs Iso p=0.018; Sct vs Iso p=0.291). Stimulation of the cells with isoproterenol was used as a positive control and induced UCP1 expression to a similar level as secretin. These results demonstrate that secretin cannot only acutely activate UCP1-mediated respiration in brown adipocytes. It can also increase UCP1 expression in these cells and thereby enhance their thermogenic potential. It should be emphasized that for this experiment secretin was used in a fivefold lower concentration than isoproterenol.

### EXAMPLE 6

### Acute Effect of Secretin on Respiration in Wildtype Mice

For the measurement of oxygen consumption wildtype mice were injected either with PBS or with secretin at a dose of 0.5 mg/kg body weight. Prior to injection basal metabolic respiration (BMR) was determined at 30° C. for 3 hours. All BMR values were highly comparable between the two groups. Irrespective of the treatment, all mice increased heat production during the first five minutes after injection (FIG. 5A). This peak most likely reflects arousal elicited by injection and a subsequent elevated excitement which lasted a few minutes after mice were released back into their cage. After ten to fifteen minutes the mice in both groups started to calm down and move less, which has been noted by continuous observation through a window in the climate chamber. The sedation is also displayed in the declining levels of heat production (A). The PBS group almost reached basal metabolic rate levels (indicated by grey background) at about 20 minutes after injection until the end of the measurement. In contrast, mice injected with secretin maintained a distinct level of elevated heat production until the end of the recording. The area under the curve (AUC) was calculated for the entire 45 minutes of the recording for both groups (B). Basal metabolic rates were subtracted for every mouse individually (mean shown in grey in figure A). Secretin stimulated heat production as demonstrated by the significant increase in the AUC (p=0.001) (FIG. 5B).

### EXAMPLE 7

### Secretin Fails to Stimulate Heat Production in UCP1 Knockout Mice

Mice show increased heat production in response to secretin injection (see example 6). To test whether this thermogenic effect is dependent on the presence of UCP1 in brown adipocytes, like already demonstrated in vitro (see example 4 and FIG. 3), calorimetric measurements were conducted in UCP1 knockout mice. Mice (wt and ko) of the 129/S1-UCP1 knockout strain were individually measured at 30° C. to determine BMR. Wildtype and knockout mice showed a comparable bodyweight (p=0.851) over all groups and no difference was detected in RER and HP during the BMR measurements. Statistical analysis was performed by two-way ANOVA.

After completion of BMR measurements, the thermogenic effect of secretin and PBS were determined (FIG. 6). After the first 15 minutes of arousal observed in all mice, wildtype mice injected with secretin maintained a higher rate of heat production as compared to PBS injected controls (FIG. 6A). The area under the curve (AUC) for the whole recording was highly significant different between the two treatment groups, with higher AUC in wildtype mice treated with secretin as compared to PBS (FIG. 6C; p=0.002). This result reproduced the data for 129/S6-wildtype mice after secretin injection (example 6, FIG. 5). In the UCP1 knockout mice there was no difference for heat production between secretin and PBS treated mice during the course of recording (FIG. 6B), or for the AUC of heat production (FIG. 6C; p=0.482).

This result clearly demonstrates that the thermogenic effect of secretin depends on the activation of UCP1-mediated heat production in brown adipose tissue.

### EXAMPLE 8

### Suppression of Food Intake in Mice after Intraperitoneal Injection of Secretin

Next to thermogenesis, brown fat has been proposed to function in the thermoregulatory control of feeding. An increase in body core temperature caused by BAT after initiation of food intake may trigger the termination of food intake (Himms-Hagen, 1995). In extension of this hypothesis secretin may be part of a novel endocrine gut—brown fat axis controlling meal patterns. Therefore, the effects of secretin on food intake in mice fasted overnight for 18 hours were tested. Just before refeeding, mice were intra-peritoneally injected with 5 nmol secretin solved in PBS, or PBS as the vehicle control, and food intake was monitored for the following three days.

Regarding the immediate refeeding responses (FIG. 7), the PBS injected control group showed a steep rise in cumulative food intake during the first hour, stopped eating during the second hour of refeeding, and recommenced eating thereafter. This trajectory was altered by secretin. Secretin treatment caused a significant inhibition of food intake during the first two hours of refeeding (p=0.0181, FIG. 7). Specifically, in secretin injected mice the initial steep rise in cumulative food intake during the first hour of refeeding was blunted, suggesting an acute and transient anorexigenic effect of this gut hormone.

Based on the hypothesis of thermoregulatory feeding, it was assessed whether the anorexigenic effect of secretin is mediated by the thermogenic activation of brown fat. To directly test this, the effect of secretin on refeeding in Ucp1 knockout mice was determined (FIG. 8). Notably, secretin did not inhibit refeeding in Ucp1 knockout mice (KO SCT) as compared to wildtype (WT SCT). This observation proves that Ucp1-mediated thermogenesis in brown adipocytes is required for the anorexigenic action of secretin. This observation identifies secretin as a new player in energy balance regulation defining a novel endocrine gut-brown fat-brain axis in the control of energy expenditure and food intake.

### EXAMPLE 9

### Sctr Gene Expression by Transcriptome Analysis (RNA-SEQ)

Method: For the RNA-sequencing method total RNA isolated from brown adipose tissue (BAT) samples of mice (n=4) housed under room temperature (23° C.) was subjected to transcriptome analysis by next generation sequencing (RNA-Seq) using Illumina HiSeq 2000 platform (Illumina). Sequencing libraries were prepared using the TruSeq RNA Sample Prep kit v2 (Illumina). Libraries from 4 samples were pooled into one sequencing lane and sequenced using a 50-cycle TruSeq SBS Kit v3-HS (Illumina), resulting in a depth of >25 million reads/sample. Sequenced tags were aligned to the Ensembl 75 transcriptome annotation (NCBI38/mm10 mouse genome) using Genomatix Software Suite. All genes and transcripts were assigned relative coverage rates as measured in RPKM units (‘reads per kilobase per million mapped reads’). In the search for gut hormones that directly activate brown adipose tissue (BAT), gene expression of gut hormone receptors was profiled by inquiring transcriptome data obtained from interscapular BAT tissue of mice. Among the detectable transcripts of gut hormone receptor genes in BAT, the secretin receptor (Sctr) gene stood out with the highest abundance expressed as reads per kilobase per million mapped reads (RPKM; FIG. 9A).

Result: Gene expression of receptors for gastrointestinal tract-related peptides obtained from RNA sequencing of murine brown adipose tissue. Data are shown as RPKM (reads per kilobase per million mapped reads). Expression of secretin receptor (Sctr) is significantly higher compared to gastric inhibitory polypeptide receptor (Gipr), vasoactive intestinal peptide receptor1/2 (Vipr1/2), cholecystokinin A/B receptor (Ccka/br), ghrelin receptor (Ghsr), glucagon-like peptide 1 receptor (Glp1r), and the PYY₃₋₃₆ receptor (Npy2r)

Method: RNA was extracted from cultured cells or frozen tissue samples using TRIsure, purified with SV Total RNA Isolation System, Promega and reverse transcribed using SensiFAST cDNA Synthesis Kit (BIOLINE). The resultant cDNA was analysed by qRT-PCR. Briefly, 1 μL of 1:10 diuluted cDNA and 400 nmol of each primer were mixed with SensiMix SYBR Master Mix No-ROX (Bioline). Reactions were performed in 384-well format using a lightcycler II instrument (Roche). Standard reactions containing serial diluted pooled cDNA of all samples (Pure, 1:2, 1:4, 1:8, 1:16, 1:32 and 1:64) as a template were used to establish a standard curve, from which gene expression levels of samples were calculated. The RNA abundance of each gene was normalized to a housekeeping gene. The following primers were used:

Sctr

| | | |
|---|---|---|
| F: | 5′-ATGCACCTGTTTGTGTCCTT-3′, | (SEQ ID NO: 12) |
| R: | 5′-TAGTTGGCCATGATGCAGTA-3′; | (SEQ ID NO: 13) |

Gtf2b

| | | |
|---|---|---|
| F: | 5′-TGGAGATTTGTCCACCATGA-3′, | (SEQ ID NO: 14) |
| R: | 5′-GAATTGCCAAACTCATCAAAACT-3′; | (SEQ ID NO: 15) |

Result: Among different fat depots, our qPCR analyses revealed that Sctr is highly expressed in BAT compared to white fat depots in the inguinal (iWAT) and epididymal (eWAT) region (FIG. 9B). Preferential Sctr expression in brown adipocytes was also observed in primary cultures derived from these fat depots (FIG. 9C).

### EXAMPLE 10

### Respiration Measurement in Primary Brown Adipocytes of Wildtype and UCP1−/− Mice

Method: To test whether SCT can activate thermogenesis in brown adipocytes, we measured UCP1-mediated uncoupled respiration in cultured adherent intact primary brown adipocytes, according to a protocol recently established in our lab (Li Y, Fromme T, Schweizer S, Schöttl T, Klingenspor M. EMBO Rep. 2014 October; 15(10):1069-76. doi: 10.15252/embr.201438775; Li Y, Fromme T, Klingenspor M. Meaningful respirometric measurements of UCP1-mediated thermogenesis. Biochimie. 2017 March; 134:56-61. doi: 10.1016/j.biochi.2016.12.005.). The cellular oxygen consumption rate (OCR) of primary adipocytes was determined using an XF96 Extracellular Flux Analyzer (Seahorse Bioscience). Briefly, primary adipocytes were cultured and differentiated in XF96 microplates. At day 7 of differentiation, cells were washed once with prewarmed, unbuffered assay medium (DMEM basal medium supplemented with 25 mM glucose, 31 mM NaCl, 2 mM GlutaMax and 15 mg/l phenol red, pH 7.4) (basal assay medium) and then the medium was replaced with basal assay medium containing 1-2% essentially fatty acid free bovine serum albumin (BSA), and incubated at 37° C. in a room air incubator for 1 h. The drug injections ports of the sensor cartridges were loaded with the assay reagents at 10× in basal assay medium (no BSA).

All respiration assays were performed in the presence of bovine serum albumin to buffer free fatty acid levels in the respiration medium. Basal respiration was measured in untreated cells. Coupled respiration was inhibited by oligomycin treatment (5 μM). UCP1 mediated uncoupled respiration was determined after isoproterenol or secretin stimulation. Maximum respiratory capacity was assessed after FCCP (Sigma-Aldrich) addition (1 μM). Finally, mitochondrial respiration was blocked by antimycin A (Sigma-Aldrich) (5 μM) treatment and the residual OCR was considered non-mitochondrial respiration. Oxygen consumption rates were automatically calculated by the Seahorse XF-96 software. Data were exported and reconstructed in GraphPad Prism 6.0 software.

Result: It was observed that SCT (10 nM) is as potent as ISO (500 nM), a β-adrenergic receptor agonist, in stimulating UCP1-mediated thermogenesis, even at a 50-fold lower concentration (FIG. 10A). Primary brown adipocytes from UCP1 knockout mice did not respond to SCT (10 nM) or ISO (500 nM) (FIG. 10B). This is further demonstrated by quantification of SCT and ISO induced leak respiration (FIG. 10C).

Cells were pretreated with propranolol (1 μM) before bioenergetic profiling. The thermogenic effect of SCT was independent on β-adrenergic receptor signaling, since pretreatment of cells with propranolol, a nonselective β-adrenergic receptor antagonist, did not attenuate SCT stimulated respiration while blocking the effect of ISO, a beta-adrenergic receptor agonist, in a dose dependent manner (FIG. 10D).

Cells were reverse transfected with small interfering RNAs (siRNAs) targeting SctR (#1CCUGCUGAUCCCUCUCUUU (SEQ ID NO: 8); #2CCCUGUCCAACUUCAUCAA (SEQ ID NO: 9); #3CCAUCGUGAUCAAUUUCAU (SEQ ID NO: 10)) and non-targeting control siRNA (UUUGUAAUCGUC GAUACCC (SEQ ID NO: 11)) as described previously (Li Y, Fromme T, Klingenspor M. Meaningful respirometric measurements of UCP1-mediated thermogenesis. Biochimie. 2017 March; 134:56-61. doi: 10.1016/j.biochi.2016.12.005.) before bioenergetic profiling. The thermogenic effect of SCT depends on SCTR, as siRNA-mediated downregulation of receptor expression, blunts the effects of SCT-induced increase in oxygen consumption (FIG. 10E).

Cells were pretreated with H89 (50 μM) before bioenergetic profiling. Pretreatment of cells with H89, a selective inhibitor of Protein Kinase A, completely blocked the thermogenic effect of SCT (FIG. 10F), demonstrating that SCT stimulated UCP1-dependent thermogenesis acts through activation of lipolysis mediated by the canonical cAMP-PKA pathway.

### EXAMPLE 11

### Plasma Secretin Levels in Response to Fating and Refeeding

Blood was collected at the time the mouse was killed. Plasma SCT levels were determined by ELISA using a kit system (Cloud-Clone CEB075Mu) following the manufacturer's instructions. Concentrations were calculated using a standard curve generated by SCT standards included in the kit. The primary stimulus for periprandial SCT release into circulation is gastric acid delivered into the duodenal lumen. Accordingly, SCT release is sensitive to the nutritional status of laboratory mice. We found that plasma secretin levels were decreased by fasting (18 hours) and increased significantly within 1 hour after refeeding (FIG. 11).

1. A modulator of the secretin receptor for use in a method for the prevention and/or treatment of a disease or disorder of energy homeostasis, wherein
(a) said modulator is a secretin receptor agonist and said disease or disorder is obesity, dyslipidemia, diabetes, insulin resistance, hyperglycemia, high blood pressure or metabolic syndrome, whereby the secretin receptor agonist increases non-shivering thermogenesis in brown adipocytes and/or increases the expression of uncoupling protein 1 (UCP1) in brown adipocytes and/or decreases food intake in a UCP1-dependent manner resulting in the prevention and/or treatment of said disease or disorder; or
(b) said modulator is a secretin receptor antagonist and said disease or disorder is cachexia.

2. The modulator of the secretin receptor for use according to claim 1, wherein the modulator of the secretin receptor is comprised in a pharmaceutical composition.

3. The modulator of the secretin receptor for use according to claim 1 or 2, wherein said modulator of the secretin receptor is to be co-administered with at least one other pharmaceutically active agent.

4. The modulator of the secretin receptor for use according to claim 3, wherein in the case of the modulator being a secretin receptor agonist said at least one other pharmaceutically active agent is selected from the group consisting of direct or indirect sympathomimetics, atrial natriuretic peptide and ANP/BNP receptor agonists.

5. A method of increasing non-shivering thermogenesis in brown adipocytes and/or increasing the expression of uncoupling protein 1 (UCP1) in brown adipocytes, comprising the step of contacting brown adipocytes with a secretin receptor agonist, thereby increasing non-shivering thermogenesis in brown adipocytes and/or increasing the expression of uncoupling protein 1 (UCP1) in brown adipocytes.

6. A method of decreasing non-shivering thermogenesis in brown adipocytes comprising the step of contacting brown adipocytes with a secretin receptor antagonist, thereby decreasing non-shivering thermogenesis in brown adipocytes.

7. A method of identifying a secretin receptor agonist capable of increasing non-shivering thermogenesis in brown adipocytes and/or increasing the expression of uncoupling protein 1 (UCP1) in brown adipocytes, comprising the steps of:
(a) determining the level of non-shivering thermogenesis in brown adipocytes and/or the expression level of uncoupling protein 1 (UCP1) in brown adipocytes or obtaining established standard values of the level of non-shivering thermogenesis and/or the of the expression of uncoupling protein 1 (UCP1);
(b) contacting said adipocytes of step (a) or other brown adipocytes with a test compound;
(c) determining the level of non-shivering thermogenesis in said brown adipocytes of step (b) and/or the expression level of uncoupling protein 1 (UCP1) in said brown adipocytes of step (b) after contacting with the test compound; and
(d) comparing the level of non-shivering thermogenesis in said brown adipocytes and/or the expression level of uncoupling protein 1 (UCP1) in said brown adipocytes determined in step (c) with the level of non-shivering thermogenesis in said brown adipocytes and/or the expression level of uncoupling protein 1 (UCP1) in said brown adipocytes determined in step (a) or to standard values of the level of the level of non-shivering thermogenesis and/or the of the expression of uncoupling protein 1 (UCP1) obtained in step (a),
wherein an increase in the level of non-shivering thermogenesis in said brown adipocytes determined in step (c) as compared to said level determined or obtained in step (a) and/or an increase in the expression level of uncoupling protein 1 (UCP1) in said brown adipocytes determined in step (c) as compared to said level determined or obtained in step (a) indicates that said test compound is a secretin receptor agonist capable of increasing non-shivering thermogenesis in brown adipocytes and/or increasing the expression of uncoupling protein 1 (UCP1) in brown adipocytes.

8. A method of identifying a secretin receptor antagonist capable of decreasing non-shivering thermogenesis in brown adipocytes, comprising the steps of:
(a) determining the level of non-shivering thermogenesis in brown adipocytes;
(b) contacting said adipocytes of step (a) or other brown adipocytes with a test compound;
(c) determining the level of non-shivering thermogenesis in said brown adipocytes of step (b) after contacting with the test compound; and
(d) comparing the level of non-shivering thermogenesis in said brown adipocytes determined in step (c) with the level of non-shivering thermogenesis in said brown adipocytes determined in step (a),
wherein a decrease in the level of non-shivering thermogenesis in said brown adipocytes determined in step (c) as compared to said level determined in step (a) indicates that said test compound is a secretin receptor antagonist capable of decreasing non-shivering thermogenesis in brown adipocytes.

9. The method of claim 7 or 8, further comprising a step of determining prior to, simultaneously with or after any one of the preceding steps (a) to (d) whether said test compound modulates the secretin receptor.

10. Use of the secretin receptor for screening
(a) for secretin receptor agonists that increase non-shivering thermogenesis in brown adipocytes and/or increase the expression of uncoupling protein 1 (UCP1) in brown adipocytes and/or decrease food intake in a UCP1-dependent manner; and/or
(b) for secretin receptor antagonists that decrease non-shivering thermogenesis in brown adipocytes.

11. Use of the secretin receptor agonist to increase non-shivering thermogenesis in brown adipocytes and/or to increase the expression of uncoupling protein 1 (UCP1) in brown adipocytes and/or to decrease food intake in a UCP1-dependent manner for reducing body weight for cosmetic purposes.

12. Use of the secretin receptor antagonist to decrease thermogenesis in brown adipocytes for increasing body weight for cosmetic purposes.

13. The modulator of the secretin receptor for use according to any one of claims 1 to 4, the method of claim 5, or the use of claim 10 or 11, wherein the secretin receptor agonist is selected from the group consisting of a small molecule, an antibody or a fragment or derivative thereof, an antibody mimetic, an aptamer, an siRNA, an shRNA, a miRNA, a ribozyme, an antisense nucleic acid molecule, secretin or a fragment or derivative thereof, a secretin analogue, or a stimulant of secretin release.

14. The modulator of the secretin receptor for use according to any one of claims 1 to 4, or the method of claim 7, or the use of claim 10 or 12, wherein the secretin receptor antagonist is selected from the group consisting of an antibody or a fragment or derivative thereof, an antibody mimetic, an aptamer, an siRNA, an shRNA, a miRNA, a ribozyme, an antisense nucleic acid molecule, or a small molecule.


SEQUENCE LISTING (15 sequences)

SEQ ID NO: 1 - protein, 9 aa, Artificial Sequence
  white fat homing peptide motif
CKGGRAKDC

SEQ ID NO: 2 - DNA, 20 bp, Artificial Sequence
  UCPI forward primer
gtacaccaaggaaggaccga

SEQ ID NO: 3 - DNA, 20 bp, Artificial Sequence
  UCPI reverse primer
tttattcgtggtctcccagc

SEQ ID NO: 4 - DNA, 20 bp, Artificial Sequence
  Secretin receptor forward primer
atgcacctgtttgtgtcctt

SEQ ID NO: 5 - DNA, 20 bp, Artificial Sequence
  Secretin receptor reverse primer
tagttggccatgatgcagta

SEQ ID NO: 6 - DNA, 20 bp, Artificial Sequence
  TFIIB forward primer
tggagatttgtccaccatga

SEQ ID NO: 7 - DNA, 23 bp, Artificial Sequence
  TFIIB reverse primer
gaattgccaaactcatcaaaact

SEQ ID NO: 8 - RNA, 19 nt, Artificial Sequence
  siRNA targeting SctR 1
ccugcugaucccucucuuu

SEQ ID NO: 9 - RNA, 19 nt, Artificial Sequence
  siRNA targeting SctR 2
cccuguccaacuucaucaa

SEQ ID NO: 10 - RNA, 19 nt, Artificial Sequence
  siRNA targeting SctR 3
ccaucgugaucaauuucau

SEQ ID NO: 11 - RNA, 19 nt, Artificial Sequence
  Non targeting control siRNA
uuuguaaucgucgauaccc

SEQ ID NO: 12 - DNA, 20 bp, Artificial Sequence
  Sctr forward primer
atgcacctgtttgtgtcctt

SEQ ID NO: 13 - DNA, 20 bp, Artificial Sequence
  Sctr reverse primer
tagttggccatgatgcagta

SEQ ID NO: 14 - DNA, 20 bp, Artificial Sequence
  Gtf2b forward primer
tggagatttgtccaccatga

SEQ ID NO: 15 - DNA, 23 bp, Artificial Sequence
  Gtf2b reverse primer
gaattgccaaactcatcaaaact